SEQUENCE LISTING (18 sequences)

SEQ ID NO: 1 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYAFYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRQAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 2 - protein, 217 aa, Hepatitis C virus
LDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLL

SEQ ID NO: 3 - protein, 253 aa, Hepatitis C virus
LLGQADAALEKLVILHAASAASSNGIVCFAIFFIAAWHIRGRAVPLATYSYLGLWSFSLLLLALPQQAYALDTTEQGQMGLVLLVVISVFTLSPAYKTLLCRSLWWLSYLLVRAEALIHEWVPPLQARGGRDGIIWAAAIFYPGVVFDITKWLLAILGPGHLLRSVLTSTPYFVRAQALLRICAAVRHLSGGKYVQMMLLTLGRWTGTYIYDHLTPLRDWAHAGLRDLAVAVEPVVFSDMETKIITWGADTAA

SEQ ID NO: 4 - protein, 80 aa, Hepatitis C virus
DAHFLSQTKQSGENFPYLVAYQATVCARAQAPPPSWDQMWKCLIRLKPTLHGPTPLLYRLGAVQNEVTLTHPITKYIMTC

SEQ ID NO: 5 - protein, 206 aa, Hepatitis C virus
PKPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKYPEATYSRCGSGPRITPRCMVDYPYRLWHYPCTINYTIFKVRMYVGGVEHRLEAACNWTRGERCDLEDRDRSELSPLLLSTTQWQVLPCSFTTLPALSTGLIHLHQNI

SEQ ID NO: 6 - protein, 217 aa, Hepatitis C virus
MDREMAASCGGVVFVGLVFLTMSPHYKVFLARLIWWLQYFLTIAEAHLQVWIPPLNIRGGRDAIILLTCVIHPELIFDITKLLLATLGPLLVLQAGIARVPYFVRAHGLIRACVLLRKVAGGHYVQMALMKLAALTGTYLYDHLTPLQYWAHAGLRDLAVAVEPVIFSDMEIKIITWGADTAACGDIIQGLPVSARRGREILLGPADSLEGQGWRLL

SEQ ID NO: 7 - protein, 217 aa, Hepatitis C virus
MDREMAPSCGGMVFLGLVLLTLSPHYKVFLARLIRWLQYFLTIAEAHLQVWVSPLNIRGGRDAVILLTCVIHPGLIFDITKLLLATLGPLLVLQAGIARVPYFVRAHGLIRACMLLRKVAGGHYVQMALMKLAALTGTYLYDHLAPLQYWAHAGLRDLAVAVEPVIFSDMEIKIITWGADTAACGDIIQGLPVSARRGREILLGPADSLDGQGWRLL

SEQ ID NO: 8 - protein, 217 aa, Hepatitis C virus
MDREMAASCGGVVFVGLVFLTLSPHYKVFLARLIWWLQYFLTIAEAHLQVWVPPLNIRGGRDAIILLTCVIHPELIFDITKLLLATLGPLLVLQAGIARVPYFVRAHGLIRACMLLRKVAGGHYVQMALMKLAALTGTYLYDHLTPLQDWAHAGLRDLAVAVEPVIFSDTEIKIITWRADTAACGDIIQGLPVSARRGREILLGPADSLDGQGWRLL

SEQ ID NO: 9 - protein, 217 aa, Hepatitis C virus
MDREMAASCGGMVFVGLVFLTLSPHYKVFLARLIWWLQYFLTIAEAHLQVWVPPLNIRGGRDAIILLTCVIHPELIFDITKLLLATLGPPLVLQAGIARVPYFVRAHGLIRACMLLRKVAGGHYVQMALMKLAALTGTYLYDHLTPLQHWAHAGLRGLAVAVEPVIFSDMEIKIITWGADTATCGDFIQGLPVSARRGREILLGPADSLEGQGWRLL

SEQ ID NO: 10 - protein, 1136 aa, Border disease virus
EGAGMTYWEGLDLQFTLLVMITASLLVARRDVTTYPLIITVIALKTTWVNSGPGIDAAIATITTGLLMWTFISDYYKYKQWTQFLISIVSGIFLIRTLKWIGGLELHAPELPSYRPLFFILTYLISAAIVTRWNLDIAGVLLQCVPTILMVLTLWADLLTLILILPTYELAKLYYLKGVKNGMERNWLGRITYKRVSDVYEIDESQEAVYLFPSKQKEGTITGGLLPLIKAILISCISSKWQCFYLLYLVVEVSYYLHKKIIEEVAGGTNLISRLVAALLEVNWRFDNEETKGLKKFYLISGQVKNLIIKHKVRNEVVAHWFNEEEVYGMPKLVSVVKAATLNRSRHCILCTVCESRDWKGETCPKCGRFGPSLSCGMTLSDFEERHYKKIFIREDQSDGPFREEYKGYLQYKARGQLFLRNLPILATKVKLLLVGNLGSEVGDLEHLGWILRGPAVCKKIIDHERCHVSIMDKLTAFFGIMPRGTTPRAPIRFPTSLLRIRRGLETGWAYTHQGGISSVDHVTAGKDLLVCDSMGRTRVVCQSNNRMTDETEYGVKTDSGCPEGARCYVFNPEAVNISGTKGAMVHLQKTGGEFTCVTASGTPAFFDLKNLKGWSGLPIFEASSGRVVGRVKVGKNEESKPTKLMSGIQTVSKSTTDLTDMVKKITTMNRGEFKQITLATGAGKTTELPRAVIEEIGRHKRVLVLIPLRAAAESVYQYMRQKHPSIAFNLRIGEMKEGDMATGITYASYGYFCQMPQPKLRAAMVEYSYIFLDEYHCATPEQLAIIGKIHRFSEQLRVVAMTATPAGTVTTTGQKHPIEEFIAPEVMKGEDLGSEFLEIAGLKIPTEEMKGNMLVFVPTRNMAVETAKKLKAKGYNSGYYYSGEDPANLRVVTSQSPYVVVATNAIESGVTLPDLDVVVDTGLKCEKRIRLSSKMPFIVTGLKRMAVTIGEQAQRRGRVGRVKPGRYYRSQETAVGSKDYHYDLLQAQRYGIEDGINITKSFREMNYDWSLYEEDSLMITQLEILNNLLISEELPVAVKNIMARTDHPEPIQLAYNSYEVQVPVLFPKIRNGEVTDSYDSYSFLNARKLGDDVPAYVYATEDEDLAVELLGMDWPDPGNQGTVETGRALKQVTGL

SEQ ID NO: 11 - protein, 1140 aa, Classical swine fever virus
GKIDGGWQRLPETSFDIQLALTVIVVAVMLLAKRDPTTVPLVITVATLRTAKMTNGLSTDIAIATVSTALLTWTYISDYYRYKTWLQYLISTVTGIFLIRVLKGIGELDLHTPTLPSYRPLFFILVYLISTAVVTRWNLDIAGLLLQCVPTLLMVFTMWADILTLILILPTYELTKLYYLKEVKTGAEKGWLWKTNFKRVNDIYEVDQSGEGVYLFPSKQKTSSITGTMLPLIKAILISCISNKWQFIYLLYLIFEVSYYLHKKIIDEIAGGTNFISRLVAALIEANWAFDNEEVRGLKKFFLLSSRVKELIIKHKVRNEVMVHWFGDEEVYGMPKLVGLVKAATLSKNKHCILCTVCEDREWRGETCPKCGRFGPPMTCGMTLADFEEKHYKRIFFREDQSEGPVREEYAGYLQYRARGQLFLRNLPVLATKVKMLLVGNLGTEVGDLEHLGWVLRGPAVCKKVTEHEKCTTSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKLMSGIQTVSKSTTDLTEMVKKITTMNRGEFRQITLATGAGKTTELPRSVIEEIGRHKRVLVLIPLRAAAESVYQYMRQKHPSIAFNLRIGEMKEGDMATGITYASYGYFCQMPQPKLRAAMVEYSFIFLDEYHCATPEQLAIMGKIHRFSENLRVVAMTATPAGTVTTTGQKHPIEEFIAPEVMKGEDLGSEYLDIAGLKIPVEEMKSNMLVFVPTRNMAVETAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPMAVKNIMARTDHPEPIQLAYNSYETQVPVLFPKIKNGEVTDSYDNYTFLNARKLGDDVPPYVYATEDEDLAVELLGLDWPDPGNQGTVEAGRALKQVVGL

SEQ ID NO: 12 - protein, 1136 aa, Bovine viral diarrhea virus 2
SRINADDQSAMDPCFLLVTGLVAVLMIARREPATLPLIVSLLAIRTSGFLLPASIDITVAVVLIVLLLASYVTDYFRYKKWLQFSFGLIAGIFIIRSLKHIDQMEVPEISMPSWRPLALVLFYITSTAITTNWDIDLAGFLLQWAPTVIMMATMWADFLTLIIVLPSYELSKLYFLKNVRTDVEKNWLGKVKYRQISSVYDICDNEEAVYLFPSRHKSGSRPDFILPFLKAVLISCISSQWQLVYITYLILEITYYMHRKIIDEVSGGANFLSRLIAAIIELNWAIDDEECKGLKKLYLLSGRVKNLIVKHKVRNEAVHRWFGEEEIYGAPKVITIIKASTLNKNRHCIICTICEGKDWNGANCPKCGRQGKPITCGMTLADFEEKHYKKIFIREGCHDGLSREEYKGYIQYTARGQLFLRNLPILATKMKLLMVGNLGAEIGDLEHLGWVLRGPAVCKKITNHEKCHVNIMDKLTAFFGIIARGTTPRAPVRFPTALLKVRRGLETGWAYTHQGGISSVDHVTAGKDLLVCDSMGRTRVVCHSNNKMTDETEYGIKTDSGCPEGARCYVLNPEAVNISGTKGAMVHLQKTGGEFTCVTASGTPAFFDLKNLKGWSGLPIFEASSGRVVGRVKVGKNEDSKPTKLMSGIQTVSKNQTDLADIVKKLTSMNRGEFKQITLATGAGKTTELPRSVIEEIGRHKRVLVLIPLRAAAESVYQYMRVKYPSISFNLRIGDMKEGDMATGITYSSYRYFFQLPQPKLKTAMVEYSYIFLDEYHCATPEQLAIIGKIHRFAENLRVVAMTATPAGTVTTTGQKHPIEEFIAPEVMKGEDLGSEYLDIAGLKIPTEEMKGNMLVFVPARNMAVETAKKLKAKGYNSGYYYSGENPENLRVVTSQSPYVVVSTNAIESGVTLPDLDTVVDTGLRCEKRVRISSKMPFIVTGLKRMAVTIGEQAQRRGRVGRVKPGRYYRSQETASGSKDYHYDLLQAQRYGIEDGINVTKSFREMNYDWSLYEEDSLMITQLEVLNNLLISEDLPAAVKNIMARTDHPEPIQLAYNSYENQIPVLFPKIKNGEVTDSYENYTYLNARKLGEDVPAYVYATEDEDLAVDLLGMDWPDPGSQQVVETGRALKQVTGY

SEQ ID NO: 13 - protein, 1226 aa, Bovine viral diarrhea virus 1
DSGGQEYLGKIDLCFTTVVLIVIGLIIARRDPTIVPLVTIMAALRVTELTHQPGVDIAVAVMTITLLMVSYVTDYFRYKKWLQCILSLVSAVFLIRSLIYLGRIEMPEVTIPNWRPLTLILLYLISTTIVTRWKVDVAGLLLQCVPILLLVTTLWADFLTLILILPTYELVKLYYLKTVRTDTERSWLGGIDYTRVDSIYDVDESGEGVYLFPSRQKAQGNFSILLPLIKATLISCVSSKWQLIYMSYLTLDFMYYMHRKVIEEISGGTNIISRLVAALIELNWSMEEEESKGLKKFYLLSGRLRNLIIKHKVRNETVASWYGEEEVYGMPKIMTIIKASTLSKSRHCIICTVCEGREWKGGTCPKCGRHGKPITCGMSLADFEERHYKRIFIREGNFEGMCSRCQGKHRRFEMDREPKSARYCAECNRLHPAEEGDFWAESSMLGLKITYFALMDGKVYDITEWAGCQRVGISPDTHRVPCHISFGSRMPFRQEYNGFVQYTARGQLFLRNLPVLATKVKMLMVGNLGEEIGNLEHLGWILRGPAVCKKITEHEKCHINILDKLTAFFGIMPRGTTPRAPVRFPTSLLKVRRGLETAWAYTHQGGISSVDHVTAGKDLLVCDSMGRTRVVCQSNNRLTDETEYGVKTDSGCPDGARCYVLNPEAVNISGSKGAVVHLQKTGGEFTCVTASGTPAFFDLKNLKGWSGLPIFEASSGRVVGRVKVGKNEESKPTKIMSGIQTVSKNRADLTEMVKKITSMNRGDFKQITLATGAGKTTELPKAVIEEIGRHKRVLVLIPLRAAAESVYQYMRLKHPSISFNLRIGDMKEGDMATGITYASYGYFCQMPQPKLRAAMVEYSYIFLDEYHCATPEQLAIIGKIHRFSESIRVVAMTATPAGSVTTTGQKHPIEEFIAPEVMKGEDLGSQFLDIAGLKIPVDEMKGNMLVFVPTRNMAVEVAKKLKAKGYNSGYYYSGEDPANLRVVTSQSPYVIVATNAIESGVTLPDLDTVIDTGLKCEKRVRVSSKIPFIVTGLKRMAVTVGEQAQRRGRVGRVKPGRYYRSQETATGSKDYHYDLLQAQRYGIEDGINVTKSFREMNYDWSLYEEDSLLITQLEILNNLLISEDLPAAVKNIMARTDHPEPIQLAYNSYEVQVPVLFPKIRNGEVTDTYENYSFLNARKLGEDVPVYIYATEDEDLAVDLLGLDWPDPGNQQVVETGKALKQVTGL

SEQ ID NO: 14 - protein, 253 aa, Hepatitis G virus
AFPLALLMGISATRGRTSVLGAEFCFDATFEVDTSVLGWVVASVVAWAIALLSSMSAGGWRHKAVIYRTWCKGYQAIRQRVVRSPLGEGRPAKPLTFAWCLASYIWPDAVMMVVVALVLLFGLFDALDWALEEILVSRPSLRRLARVVECCVMAGEKATTVRLVSKMCARGAYLFDHMGSFSRAVKERLLEWDAALEPLSFTRTDCRIIRDAARTLSCGQCVMGLPVVARRGDEVLIGVFQDVNHLPPGFVPT

SEQ ID NO: 15 - protein, 1136 aa, Pestivirus Reindeer-1
DTAETVEAGSIDLQFILLVAITASLLVARRDATTYPLIITVVALRTTWVNSGPGLDVAIASLTTGLLMWTFISDYHKYKRWLQFSISIVSGIFIIRTLKWVGGAELSVPELPSYRPLFFILTYLISTAVVTRWNLDIAGALLQCVPTLLMVMTLWADLLTLVLILPTYELTKLYYLKGVKQGMERNWLGKVSYKRVSDIYEIDESQEAVYLFPSKQKGGSITGGLLPLLKAILISCVSSKWQCFYLLYLVIELSYYLHKKIIEKIAGGTNLISRLIAALLEVTWEFDDKETRGLKKFYLLSSRVRSLIIKHKVRNEVMVHWFEEEEVYGMPKLVSVVKSATLNKSRHCILCTVCENREWKGETCPKCGRCGPPVSCGMTLSDFEERHYKRIFVREDQSEGPFREEYKGYLQYRARGQLFLRNLPILATKVKLLLVGNLGSEVGDLEHLGWILRGPAVCKKITDHEKCHVSIVDKLTAFFGIMPRGTTPRAPIRFPTSLLRIRRGLETGWAYTHQGGISSVDHVTAGKDLLVCDSMGRTRVVCQSNNKMTDETEYGVKTDSGCPEEQRCYVFNPEAVNISGTKGAMVHLQKTGGEFTCVTASGTPAFFDLKNLKGWSGLPIFEASSGRVVGRVKVGKNEESKPTKLMSGIQTVSKSTTDLTEMVKKITSMNRGEFKQITLATGAGKTTELPRAVIEEIGRHKRVLVLIPLRAAAESVYQYMRQKHPSIAFNLRIGEMKEGDMATGITYASYGYFCQMPQPKLRAAMVEYSYIFLDEYHCATPEQLAIIGKIHRFSEQLRVVAMTATPAGTVTTTGQKHPIEEFIAPEVMKGEDLGSEFLEIAGLKIPTEEMKGNMLVFVPTRNMAVETAKKLKAKGYNSGYYYSGEDPANLRVVTSQSPYVVVATNAIESGVTLPDLDVVVDTGLKCEKRIRLSSKMPFIVTGLKRMAVTIGEQAQRRGRVGRVKPGRYYRSQETAVGSKDYHYDLLQAQRYGIEDGINITKSFREMNYDWSLYEEDSLMITQLEILNNLLISEELPIAVKNIMARTDHPEPIQLAYNSYEVQVPVLFPKIRNGEVTDTYDTYTFLNARKLGDDVPAYVYSTEDEDLAVELLGLDWPDPGNQTTAETGRALKQVTGL

SEQ ID NO: 16 - protein, 208 aa, Hepatitis GB virus B
FDTEIIGGLTIPPVVALVVMSRFGFFAHLLPRCALVNSYLWQRWENWFWNVTLRPERFFLVLVCFPGATYDALVTFCVCHVALLCLTSSAASFFGTDSRVRAHRMLVRLGKCHAWYSHYVLKFFLLVFGENGVFFYKHLHGDVLPNDFASKLPLQEPFFPFEGKARVYRNEGRRLACGDTVDGLPVVARLGDLVFAGLAMPPDGWAIT

SEQ ID NO: 17 - protein, 148 aa, Bovine viral diarrhea virus 2
RVGRVKPGRYYRSQETATGSKDYHYDLLQAQRYGIEDGINVTKSFREMNYDWSLYEEDSLMITQLEVLNNLLISEDLPAAVKNIMARTDHPEPIQLAYNSYENQVPVLFPKIKNGEVTDSYENYTYLNARKLGEDVPAYVYATEDEDL

SEQ ID NO: 18 - protein, 70 aa, Hepatitis C virus subtype 1b
AAWYVKGRLVPGAAYAIYGVWPLLLLLLALPPRAYAMDREVAASCGGAVFILLVLLTMTPHYKLFLAKLI

(19) United States
(12) Patent Application Publication
(10) Pub. No.: US 2009/0233266 A1
(43) Pub. Date: Sep. 17, 2009

(54) STRUCTURE OF THE HEPATITIS C VIRUS NS2 PROTEIN

(75) Inventors: Charles Rice, New York, NY (US); Joseph Marcotrigiano, New Brunswick, NJ (US); Ivo C. Lorenz, Brooklyn, NY (US)

Correspondence Address: THOMPSON COBURN LLP, ONE US BANK PLAZA, SUITE 3500, ST LOUIS, MO, 63101, US

(73) Assignee: The Rockefeller University, New York, NY (US)

(21) Appl. No.: 11/817,782

(22) PCT Filed: Mar. 9, 2006
(86) PCT No.: PCT/US06/08535

§ 371 Date: Aug. 4, 2008

Related U.S. Application Data
(60) Provisional application No. 60/659,908, filed on Mar. 10, 2005.

(51) Int. Cl. C12Q 1/70; C07K 1/00

(52) U.S. Cl. 435/5; 530/402

(57) ABSTRACT

The present invention provides a crystallized C-terminal domain of an NS2 protein of hepatitis C virus, methods of producing the same and methods of use thereof. The present invention also relates to structural elements of the C-terminal domain of hepatitis C virus NS2 protein, and methods of inhibiting hepatitis C virus infection, replication and/or pathogenesis, by interacting with the same.

### FIELD OF THE INVENTION

This invention provides crystallized C-terminal domains of a nonstructural protein 2 (NS2) of hepatitis C virus, methods of producing the same and methods of use thereof. The present invention also relates to structural elements of the C-terminal domain of hepatitis C virus NS2 protein, the enzymatic mechanism of the same, and methods of inhibiting hepatitis C virus infection and/or pathogenesis, by interacting with, or interfering with the same.

### BACKGROUND OF THE INVENTION

Hepatitis C virus (HCV) is a causal agent of chronic liver disease in humans, afflicting more than 170 million people worldwide. Chronic infection with HCV can progress to liver cirrhosis and hepatocellular carcinoma, and HCV is currently the major cause for liver transplantation. Antiviral therapy using a combination of pegylated interferon-? and ribavirin leads to a sustained response in only about 50% of patients, and no vaccine is available so far.

HCV forms a unique genus, hepacivirus, within a family of small enveloped RNA viruses, the Flaviviridae. HCV has a broad sequence diversity and is divided into 6 distinct genotypes and more than 30 subtypes. The virus forms quasi-species, with many closely related sequences existing in parallel in the infected individual.

The genome of HCV consists of a single positive-stranded RNA molecule of about 9.6 kb in length. It contains one long open reading frame, leading to the production of a large polyprotein precursor of roughly 3000 amino acids, which is co- and post-translationally cleaved into at least 10 proteins by host cellular and virus-encoded proteases. In addition, the core region contains an alternate reading frame coding for the expression of the F protein, a small protein of unknown function. The ORF is flanked by 5′ and 3′ untranslated regions (UTR), which are involved in RNA replication and translation initiation. Three structural proteins (core, envelope proteins E1 and E2), which are the presumed components of the mature virion, lie at the N-terminus of the polyprotein, followed by a small hydrophobic protein, p7. The nonstructural proteins (NS2, NS3, NS4A, NS4B, NS5A, and NS5B) are located in the C-terminal two thirds of the polyprotein.

NS3, NS4A, NS4B, NS5A, and NS5B are part of the viral replication complex, which propagates the multiplication of the viral genome. Distinct roles have been elucidated for some of the nonstructural proteins: NS3 has helicase and protease functions; while NS5B is the viral RNA-dependent RNA polymerase. NS4A was shown to be a cofactor for the NS3 protease. The functions of the remaining nonstructural proteins, such as NS4B, and NS5A, are less well characterized.

Research on the viral life cycle of HCV has been hampered by its limited replication in tissue culture systems as well as by the lack of small animal models. Recent progress has been made by the development of full-length and subgenomic viral replicons, the latter spanning the NS3 to NS5B region. The replicons consist of the HCV internal ribosomal entry site (IRES) driving the expression of a selectable marker, followed by the IRES of encephalomyocarditis virus (EMCV) placed upstream of the genes encoding the HCV polypeptide. This allows the selection of stably replicating populations of Huh7 cells, a human hepatoma cell line. Additionally, adaptive amino acid substitutions in NS3 and NS5A have been identified that enable efficient replication of the native HCV genome in Huh7 cells.

NS2 (217 amino acids, 23 kDa), together with the immediately downstream 180 amino acids of NS3, contains an autoprotease activity that cleaves the HCV polyprotein at the NS2/3 junction. NS2-3 protease activity was shown to be necessary for the in vivo infectivity of full-length HCV genomes. Moreover, inhibition of the cleavage at the NS2/3 junction abolishes replication of viral RNA the full-length replicon. However, NS2 is dispensable for the replication of subgenomic HCV replicons and may thus not be part of the replication complex. Given the small size of the HCV genome, which has to encode all the relevant factors required for viral replication and pathogenesis, it is likely that NS2 has other functions before and/or after the autoproteolytic cleavage from NS3. Obtaining structural information of NS2 will help in understanding the molecular mechanism of the autoprotease function, and it will also aid the development of antiviral therapies against HCV infection.

### SUMMARY OF THE INVENTION

The invention provides, in one embodiment, a crystallized C-terminal domain of an NS2 protein of hepatitis C virus, wherein the crystal effectively diffracts X-rays for the determination of the atomic coordinates of the domain to a resolution of greater than 5.0 Angstroms, and wherein said crystal has a space group of P2₁, with unit cell dimensions of a=61.23 Å, b=67.27 Å, c=108.87 Å and ?=?=90°, ?=105.82°.

In another embodiment, this invention provides a crystallized C-terminal domain of an NS2 protein of hepatitis C virus, wherein the crystal effectively diffracts X-rays for the determination of the atomic coordinates of the domain to a resolution of greater than 5.0 Angstroms, and wherein said crystal has a space group of P2₁, with unit cell dimensions of a=109.81 Å, b=68.82 Å, c=125.16 Å, and ?=?=90°, ?=1105.88°.

In another embodiment, the invention provides a computer readable data storage material encoded with computer readable data comprising structure coordinates of Table 1.

In another embodiment, this invention provides a crystallized C-terminal domain of an NS2 protein of hepatitis C virus, wherein the C-terminal domain of an NS2 protein of hepatitis C virus has secondary structural elements that include two alpha helices in the N-terminal subdomain, designated as alpha helices H1 and H2, and an antiparallel beta sheet in the C-terminal subdomain, consisting of one beta strand in the linker arm between the N-terminal and the C-terminal subdomains, termed b1, and three beta strands in the C-terminal subdomain, named b2, b3, and b4.

In another embodiment, this invention provides a crystallized C-terminal domain of an NS2 protein of hepatitis C virus, which shows a papain-like active site that mediates the autoproteolytic cleavage after the NS2 carboxy-terminus. The amino acid residues in the active site comprise histidine 143, glutamate 163, cysteine 184, and the C-terminal leucine 217. The numbering is relative to the actual amino-terminus of NS2.

In another embodiment, this invention provides a crystallized C-terminal domain of an NS2 protein of hepatitis C virus, which forms a dimer that contains a domain swap of its carboxy-terminal subdomain. The active site of each protein subunit is composed of amino acid residues from both monomers, with the histidine and glutamate from one molecule, and the cysteine and carboxy terminus from the other.

In another embodiment, the invention provides a method of using a crystal of this invention in an inhibitor screening assay, the method comprising selecting a potential inhibitor by performing rational drug design with the three-dimensional structure determined for the crystal, wherein selecting is performed in conjunction with computer modeling, contacting the potential inhibitor with an C-terminal domain of an NS2 protein of hepatitis C virus and detecting the ability of the potential inhibitor for inhibiting infection or replication of a hepatitis C virus.

According to this aspect of the invention, and in one embodiment, the inhibitor interferes with the autoproteolytic cleavage mediated by the C-terminal domain of an NS2 protein. In another embodiment, the inhibitor interferes with dimerization of the NS2 proteins of the virus. In another embodiment, the inhibitor interferes with the membrane association of the N-terminal subdomain of NS2.

In another embodiment, this invention provides a method of growing a crystallized C-terminal domain of an NS2 protein, comprising growing the crystal by vapor diffusion using a reservoir buffer containing 100 mM Tris pH 8.5, 0.8 M ammonium acetate, 0.25 M lithium chloride and 12% (w/v) polyethylene glycol 3350, at 4° C.

In another embodiment, this invention provides a method for identifying a test compound that interferes with the autoproteolytic cleavage mediated by the C-terminal domain of an NS2 protein, the method comprising:
(a) providing in vitro conditions wherein an NS2/NS3/NS4A protein, or fragment thereof comprising junctional sequences between NS2, NS3 and NS4 is produced, such that said junctional sequences are intact;
(b) contacting said NS2/NS3/NS4A protein, or fragment thereof with a test compound, under conditions and for a time sufficient for autoproteolytic cleavage of a junction between NS2 and NS3 of said protein or fragment thereof to occur;
(c) contacting said NS2/NS3/NS4A protein, or fragment thereof without said test compound, under conditions and for a time sufficient for autoproteolytic cleavage of a junction between NS2 and NS3 of said protein or fragment thereof to occur;
(d) detecting whether said junctional sequences in (b) versus (c) are intact,
   whereby a decrease or absence in intact junctional sequences as detected in (c) as compared to (b) indicates that the test compound interferes with the autoproteolytic activity of a C-terminal domain of an NS2 protein.

In another embodiment, this invention provides a method for identifying a test compound that inhibits or prevents hepatitis C viral infection or pathogenesis, the method comprising:
a) contacting a cell in culture harboring replicating hepatitis C virus RNA or infected with a hepatitis C virus in culture with a test compound, under conditions and for a time sufficient to permit the dimerization of NS2 proteins of said virus;
b) contacting a cell in culture harboring replicating hepatitis C virus RNA or infected with hepatitis virus C virus in the absence of said agent, under conditions and for a time sufficient to permit the dimerization of said NS2 protein; and
c) comparing viral infection or pathogenic effects on cells cultured in (a) versus (b),
   whereby a decrease or absence of viral infection or pathogenic effects on cells detected in (a) as compared to (b) indicates that the test compound inhibits or prevents hepatitis C viral infection or pathogenesis.

In another embodiment, this invention provides a method for inhibiting hepatitis C viral infection or pathogenesis, comprising contacting an C-terminal domain of an NS2 protein, or a fragment thereof, of hepatitis C virus with an agent that interferes with the membrane association of said C-terminal domain of an NS2 protein.

In another embodiment, this invention provides a method for inhibiting hepatitis C viral infection or pathogenesis, comprising contacting an C-terminal domain of an NS2 protein, or a fragment thereof, of hepatitis C virus with an agent that interferes with the active site of NS2.

### BRIEF DESCRIPTION OF THE DRAWINGS

FIG. 1 schematically depicts an overview of the NS2 structure. A. Ribbon diagram of the structure of an NS2 dimer, with one monomer highlighted, the other not. Secondary structure elements are labeled as described in the text, and the amino- and carboxy-termini of each monomer are indicated. The two subunits of the dimer make extensive contacts, with a total buried surface area of roughly 1100 Å². B. A 90° rotation around the horizontal axis, showing a ‘top’ view of the NS2 dimer. C. A 90° back-rotation around the horizontal axis, and a 90° rotation around the vertical axis showing a ‘side’ view of the NS2 dimer.

FIG. 2 schematically depicts the active site of NS2, which mediates the autoproteolytic cleavage at its carboxy-terminus. The amino acid residues of the catalytic triad, consisting of histidine 143, glutamate 163 and cysteine 184, as well as the carboxyterminal leucine 217, are shown as ball-and-stick drawings. Note that the active site is composed of histidine 143 and glutamate 163 from one molecule of the dimer (chain A, drawn in light grey), and cysteine 184 and the carboxy-terminal leucine 217 from the other molecule (chain B, drawn in dark grey). Amino acid residues are numbered according to the hepatitis C virus polyprotein sequence.

FIG. 3 demonstrates molecular surface properties of NS2. A. Solvent-accessible surface of NS2 colored according to electrostatic potential: acidic (light), neutral (white), and basic (dark), oriented as in FIG. 1A. B. A 90° rotation around the horizontal axis, showing the electrostatic potential the ‘top’ surface of the NS2 dimer. This region of the NS2 dimer is highly hydrophobic, suggesting that this side of the molecule may be peripherally inserted into a cellular membrane. C. Solvent-accessible surface of one NS2 molecule, colored according to sequence conservation. Grey corresponds to 95% or greater conservation, light grey to 75-95% conservation, and white to less than 75% conservation. The other molecule of the dimer is drawn as ball-and-stick model with the C-? trace shown as ‘worm’ representation.

FIG. 4 depicts a model of the electrostatic potential at the surface of the NS2 dimer positioned relative to the membrane of the endoplasmic reticulum (ER). The view is according to the orientation in FIG. 1A. The hydrophobic part of helix H2 from each subunit is peripherally inserted into the lipid bilayer, with basic amino acids on the ‘side’ of the helix involved in neutralizing the charge of acidic lipid head groups in the membrane. This model places the amino-termini of the NS2 dimer close to the ER membrane, where the putative upstream transmembrane segments of the protein would precede the protease domain.

### DETAILED DESCRIPTION OF THE INVENTION

This invention provides, in one embodiment, crystallized C-terminal domains of a non-structural NS2 protein of hepatitis C virus, methods of producing the same and methods of use thereof. The present invention provides, in other embodiments, structural elements of the C-terminal domain of hepatitis C virus NS2 protein, and, in other embodiments, the invention provides methods of inhibiting hepatitis C virus infection and/or pathogenesis, by interacting with, or interfering with the same.

In another embodiment, this invention provides a method of growing a crystallized C-terminal domain of an NS2 protein, comprising growing the crystal by vapor diffusion using a reservoir buffer containing 100 mM Tris pH 8.5, 0.8 M ammonium acetate, 0.25 M lithium chloride, and 12% (w/v) polyethylene glycol 3350, at 4° C.

The HCV nonstructural protein NS2 contains a cysteine protease activity that processes the HCV polyprotein precursor through autocleavage at the junction between nonstructural proteins NS2 and NS3.

The C-terminal domain of NS2 contains a helix-turn-helix motif at its aminoterminus, which promotes association with cellular membranes. The remainder consists of the active site of the autoprotease that mediates cleavage at the carboxyterminus in a papain-like manner.

The C-terminal domain of NS2 forms a dimer with a domain swap of its carboxyterminal subdomains. Dimerization is essential for establishing the active site of the protease and thus for the autoproteolytic cleavage at the carboxyterminus of the protein.

This invention provides, in one embodiment, a mechanistic understanding of the role of NS2 in these processes.

The C-terminal domain of NS2 has six essentially identical monomers per asymmetric unit packed together as three dimers via contacts in the aminoterminal helices of the molecule. The larger form of the unit cell contains twelve essentially identical monomers per asymmetric unit, packed together as two hexamers formed by three dimers each. The amino-terminal domain of NS2 consists of two ?-helices (H1 and H2) connected by a short turn, and four ?-strands (b1 through b4). Strand b1 from one molecule in the dimer forms a b-sheet with strands b2, b3, and b4 from the other molecule.

In one embodiment, NS2 has an amino acid sequence such as that disclosed in Genbank Accession Number: AAB66324, NP_{—}751923, AAV35990, AAA99036, AAA45615, AAD50789, AAD50788, AAD50787, AAD50786, or a sequence homologous thereto.

Crystallographic analysis was conducted herein, of the C-terminal domain of NS2. The crystal form produced is described hereinbelow.

In one embodiment, this invention provides, a crystallized C-terminal domain of an NS2 protein of hepatitis C virus, wherein the crystal effectively diffracts X-rays for the determination of the atomic coordinates of the domain to a resolution of greater than 5.0 Angstroms, and wherein said crystal has a space group of P2₁, with unit cell dimensions of either a=61.23 Å, b=67.27 Å, c=108.87 Å and ?=?=90?, b=105.82 Å; or a=109.81 Å, b=68.82 Å, c=125.16 Å, and ?=?=90°, ?=105.88°.

In another embodiment, the invention provides a computer readable data storage material encoded with computer readable data comprising structure coordinates of Table 1.

In another embodiment, this invention provides a crystallized C-terminal domain of an NS2 protein of hepatitis C virus, wherein the C-terminal domain of an NS2 protein of hepatitis C virus has secondary structural elements that include two alpha helices, designated H1 and H2, and four beta strands, designated as strands b1, b2, b3 and b4.

In another embodiment, this invention provides a crystallized C-terminal domain of an NS2 protein of hepatitis C virus, which shows a papain-like active site that mediates the autoproteolytic cleavage at the carboxy-terminus. The amino acid residues in the active site comprise histidine 143, glutamate 163, cysteine 184, and the C-terminal leucine 217.

In another embodiment, this invention provides for the crystallization of the protein to obtain other forms, such as, having different space group and/or unit cell dimensions. In one embodiment, the structural data for the crystals of this invention may be obtained by methods known to one skilled in the art, such as those exemplified herein, and may include other structural methods such as, for example, NMR.

The structures of the C-terminal domain of an NS2 proteins of the invention provide especially meaningful guidance for the development of drugs to target and inhibit its autoproteolytic activity, or in another embodiment, inhibit dimerization, or in another embodiment, inhibit association with cellular membranes, which, in other embodiment, represents various means to regulate NS2 activity.

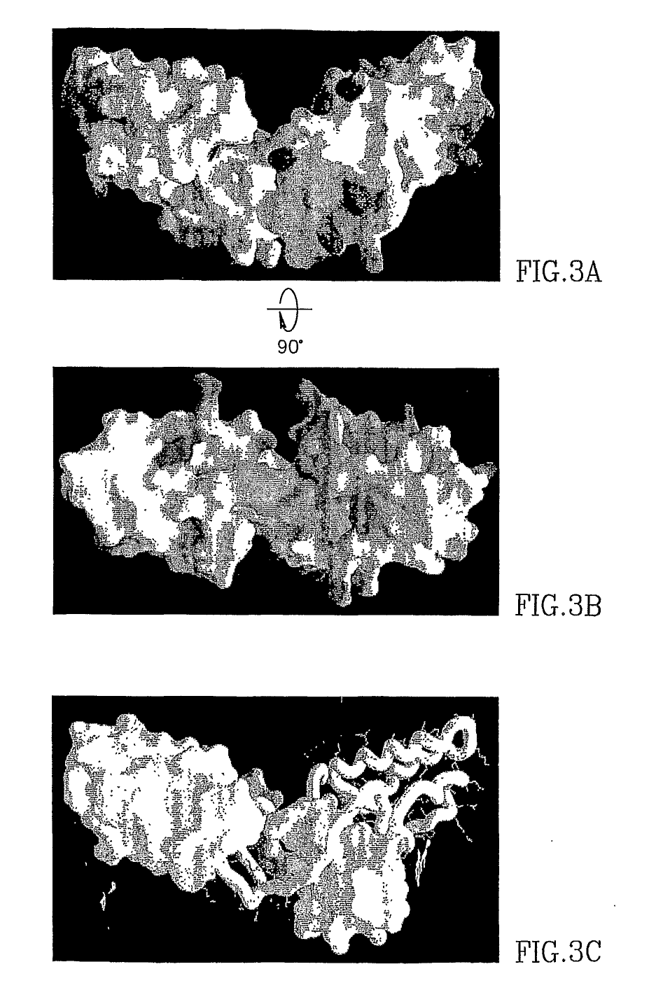

Structural analysis provided an understanding of how the C-terminal domain of an NS2 protein interacts with RNA or protein. Sequence alignments of NS2 domain I regions from 30 HCV genotype reference sequences, as well as the sequence of the related GB virus B NS2, shows the significant overall surface conservation of NS2, and highlights a large patch of conserved residues that represents a molecular interaction surface (FIG. 3C).

The structure coordinates provided can be used to solve the structure of other NS2 proteins, NS2 mutants, or N-terminal domains thereof, co-complexes with the same, or of the crystalline form of any other protein with significant amino acid sequence homology thereof.

In one embodiment, crystallization of the C-terminal domain of NS2, can be conducted as described and exemplified herein, with crystallized products utilized in the methods of this invention, as further described. In one embodiment, the crystals of this invention may be used for compound ‘soaking’ in ‘co-crystallization’ experiments. According to this aspect of the invention, and in one embodiment, an agent of interest, such as, for example, and in one embodiment, a small molecule, may be added to the protein prior to crystallization or added to pre-formed crystals to determine the location of the binding of the small molecule on the NS2 protein by structural solution of the complex.

In one embodiment, the invention provides for a protein, or a fragment thereof, which has a structure which roughly approximates that of the C-terminal domain of NS2, as described herein, wherein the structure of the molecule provides for functional equivalency or correspondence with that of NS2, such as, for example, interaction with host cell proteins, as described hereinbelow, or, in another embodiment, other viral proteins. In another embodiment, the protein with similar structural characteristics will have a homologous amino acid sequence to that of the C-terminal domain of NS2, as described hereinbelow.

In another embodiment, the invention provides for crystals which include crystallized mutants of NS2, wherein, in one embodiment, the mutation results in abrogation of the autoproteolytic activity, as described herein, by histidine 143, glutamate 163, and cysteine 184.

In another embodiment, the invention provides for proteins, or in another embodiment, crystallized forms, wherein the protein comprises mutations, which result in the abrogation of dimer formation.

One embodiment for a method that may be employed for such purposes, in preparing such mutated forms of the NS2 protein, or fragments thereof, is molecular replacement. In this method, in one embodiment, an unknown crystal structure may be determined using the protein structure coordinates of the C-terminal domain of NS2 of this invention.

In one embodiment, the term “molecular replacement” refers to a method that involves generating a preliminary model of a crystal of a viral protein thought to interact with a cellular protein, whose structure coordinates are unknown, by orienting and positioning a molecule whose structure coordinates are known, such as the C-terminal domain of NS2 coordinates, within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal, as is known by those of ordinary skill in the art. Using the structure coordinates of the C-terminal domain of NS2 provided by this invention, molecular replacement can thus be used to determine the structure coordinates of, in other embodiments, a crystalline mutant or homolog of NS2, or additional crystal forms of NS2.

In another embodiment, the invention provides a method of using a crystal of this invention in an inhibitor screening assay, the method comprising selecting a potential inhibitor by performing rational drug design with the three-dimensional structure determined for the crystal, wherein selecting is performed in conjunction with computer modeling, contacting the potential inhibitor with an NS2 protein of hepatitis C virus and detecting the ability of the potential inhibitor for inhibiting infection or replication of a hepatitis C virus.

According to this aspect of the invention, and in one embodiment, the inhibitor interferes with the autoproteolytic activity to the C-terminal domain of an NS2 protein. In another embodiment, the inhibitor interferes with dimerization of NS2 proteins of the virus. In another embodiment, the inhibitor interferes with the association of NS2 with cellular membranes.

In one embodiment, the potential inhibitor is contacted with an C-terminal domain of an NS2 protein of hepatitis C virus.

Numerous computer programs are available and suitable for rational drug design and the processes of computer modeling, model building, and computationally identifying, selecting and evaluating potential inhibitors of dimerized NS2 proteins, or C-terminal domains thereof, of hepatitis C virus in the methods described herein. These include, for example, GRID (available form Oxford University, UK), MCSS (available from Molecular Simulations Inc., Burlington, Mass.), AUTODOCK (available from Oxford Molecular Group), FLEX X (available from Tripos, St. Louis. Mo.), DOCK (available from University of California, San Francisco), CAVEAT (available from University of California, Berkeley), HOOK (available from Molecular Simulations Inc., Burlington, Mass.), and 3D database systems such as MACCS-3D (available from MDL Information Systems, San Leandro, Calif.), and UNITY (available from Tripos, St. Louis. Mo. Potential agents may also be computationally designed “de novo” using such software packages as LUDI (available from Biosym Technologies, San Diego, Calif.), LEGEND (available from Molecular Simulations Inc., Burlington, Mass.), and LEAPFROG (Tripos Associates, St. Louis, Mo.). Compound deformation energy and electrostatic repulsion, may be evaluated using programs such as GAUSSIAN 92, AMBER, QUANTA/CHARMM, AND INSIGHT II/DISCOVER. These computer evaluation and modeling techniques may be performed on any suitable hardware including for example, workstations available from Silicon Graphics, Sun Microsystems, and the like. These techniques, methods, hardware and software packages are representative and are not intended to be comprehensive listing. Other modeling techniques known in the art may also be employed in accordance with this invention. See for example, N.C. Cohen, Molecular Modeling in Drug Design, Academic Press (1996) (and references therein), and software identified at internet sites including the CAOS/CAMM Center Cheminformatics Suite at http://www.caos.kun.nl/, and the NIH Molecular Modeling Home Page at http://www.fi.muni.cz/usr/mejzlik/mirrors/molbio.info.nih.gov/modeling/softwarelist/.

The agent is selected by performing rational drug design with the three-dimensional structure (or structures) determined for the crystal described herein, especially in conjunction with computer modeling and methods described above. The agent is then obtained from commercial sources or is synthesized from readily available starting materials using standard synthetic techniques and methodologies known to those of ordinary skill in the art. The agent is then assayed, in one embodiment, to determine its ability to inhibit dimerization of an NS2 protein, or an C-terminal domain thereof of hepatitis C virus, or, in another embodiment, autoproteolysis at the carboxy terminus of NS2, or in another embodiment, NS2 protein association with cellular membranes, or in another embodiment, cellular proteins, by methods well known in the art.

The agent selected or identified by the aforementioned process may be assayed to determine its ability to affect HCV infection, in one embodiment, or in another embodiment, HCV replication. The assay may be in vitro or in vivo. The compounds described herein may be used in assays, including radiolabeled, antibody detection and fluorometric, in another embodiment, for the isolation, identification, or structural or functional characterization of NS2. Such assays may include, in another embodiment, an assay, utilizing a full length NS2, or in another embodiment, a C-terminal fragment thereof, which, in another embodiment, is contacted with the agent and a measurement of the binding affinity of the agent against a standard is determined.

The assay may, according to this aspect of the invention, employ fluorescence polarization measurements. Agents, such as, in one embodiment, peptides or in another embodiment, proteins, or in another embodiment, RNA, which are expected to bind to NS2 are labeled with fluorescein. Labeled agent, or in another embodiment, peptide, or in another embodiment, protein, is then titrated with increasing concentrations of NS2, and the fluorescence polarization emitted by the labeled agent/peptide is determined. Fluorescence emission polarization is proportional to the rotational correlation time (tumbling) of the labeled molecule. Tumbling, in part, depends on the molecular volume, i.e. larger molecules have larger volume and slower tumbling which in turn gives rise to increased polarization of emitted light. If the agent/peptide associates with NS2, its effective molecular volume greatly increases, which may be evidenced by values obtained for polarization fluorescence emissions.

In one embodiment, complexes of peptides, or in another embodiment, proteins, or in another embodiment, agents, with the C-terminal domain of an NS2 protein may be studied using well-known X-ray diffraction techniques, or in another embodiment, as exemplified herein, and in another embodiment, may be refined versus 2-3 angstrom resolution X-ray data to an R value of about 0.20 or less using readily available computer software, such as X-PLOR (Yale University©, 1992, distributed by Molecular Simulations, Inc.; Blundel & Johnson, 1985, specifically incorporated herein by reference).

The design of compounds that inhibit NS2 dimerization, or that of C-terminal domains thereof and/or, in another embodiment, protein activity, according to this invention may involve several considerations. In one embodiment, the compound should be capable of physically and structurally associating with the C-terminal domain of an NS2 protein, such as, in other embodiments, by using non-covalent molecular interactions, including hydrogen bonding, van der Waals and hydrophobic interactions and the like. In another embodiment, the compound may assume a conformation that allows it to associate with the C-terminal domain of an NS2 protein, or in another embodiment, with the active site, or in another embodiment, with regions important in membrane association. In another embodiment, although certain portions of the compound may not directly participate in this association with NS2, those portions may still influence the overall conformation of the molecule, or in another embodiment, to catalytic activity, or in another embodiment, dimerization. This, in turn, may have a significant impact on potency. Such conformational requirements include the overall three-dimensional structure and orientation of the chemical entity or compound in relation to all or a portion of the binding site, in another embodiment.

In another embodiment, an inhibitor may be designed using the structure of NS2, where the inhibitors disrupt inter-subdomain contacts in NS2. In one embodiment, molecules are designed to specifically bind at the interface between the aminoterminal and carboxyterminal subdomains of the protein and inhibit function by altering molecular conformation.

In one embodiment, the term “inhibitor” refers to a molecule which affects NS2 structure, and/or, in another embodiment, function and/or, in another embodiment, activity. In one embodiment, the inhibitors obtained via this invention may also be referred to as “drugs”, in that they may be administered to a subject as part of anti-viral therapy.

The potential inhibitory activity of a chemical compound on NS2 dimerization and/or proteolytic activity may be analyzed prior to its actual synthesis and testing by the use of computer modeling techniques, as is known to those of ordinary skill in the art.

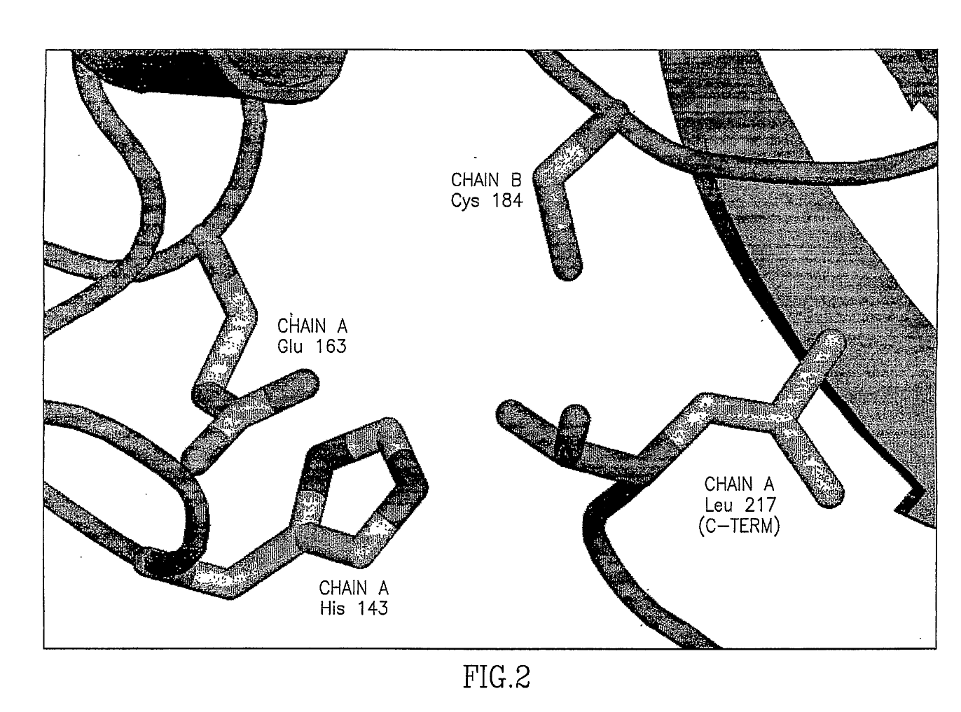

One of ordinary skill in the art may use, in other embodiments of this invention, any one of several methods to screen chemical entities or fragments for their ability to associate with NS2, or a C-terminal domain thereof, and, in another embodiment, with the active site of NS2 mediating autoproteolytic cleavage. This process may begin by visual inspection of, for example, the active site of NS2 on the computer screen based on data presented in, for example, FIG. 2. Selected fragments or chemical entities may then be positioned in a variety of orientations, or docked, within the active site, for example, and in other embodiments. Docking may be accomplished using software such as Quanta and Sybyl, followed by energy minimization and molecular dynamics with standard molecular mechanics forcefields, such as CHARMM and AMBER.

Specialized computer programs may also assist in the process of selecting fragments or chemical entities. These include, in one embodiment, the programs GRID, MCSS, AUTODOCK and DOCK.

Once suitable chemical entities or fragments have been selected, they may, in another embodiment, be assembled into a single compound. Assembly may be preceded by visual inspection of the relationship of the fragments to each other on the three-dimensional image displayed on a computer screen in relation to the structure coordinates of NS2 protein. This may be followed, in another embodiment, by manual model building using software such as Quanta or Sybyl.

Useful programs to aid one of skill in the art in connecting the individual chemical entities or fragments include, in other embodiments, CAVEAT, 3D Database systems such as MACCS-3D (MDL Information Systems, San Leandro, Calif.) and HOOK.

In another embodiment, instead of proceeding to build an agent which interacts with the NS2 protein, or a C-terminal fragment thereof, in a step-wise fashion, one fragment or chemical entity at a time as described above, the agent may be designed as a whole or “de novo” using either an empty binding site. These methods may include the use of programs such as LUDI, LEGEND and LeapFrog, each of which represents an embodiment of this invention.

In another embodiment, once a compound has been designed or selected by the above methods, the efficiency with which that compound may bind to the NS2 protein may be tested and optimized by computational evaluation. In such methods, the deformation energy of binding may be considered and agents, which interact with the NS2 protein, or a C-terminal fragment thereof, may be designed with a particular deformation energy of binding, as will be understood by one of ordinary skill in the art.

A compound designed or selected as binding to the NS2 protein may, in another embodiment, be further computationally optimized so that in its bound state it would preferably lack repulsive electrostatic interaction with the NS2 protein. Such non-complementary (e.g., electrostatic) interactions include, in other embodiments, repulsive charge-charge, dipole-dipole and charge-dipole interactions. Specifically, the sum of all electrostatic interactions between the bound agent and the NS2 protein, make, in another embodiment, a neutral or favorable contribution to the enthalpy of binding.

Specific computer software is available in the art to evaluate compound deformation energy and electrostatic interaction, and may include, in other embodiments, Gaussian 92, revision C (Frisch, Gaussian, Inc., Pittsburgh, Pa., © 1992); AMBER, version 4.0 (Kollman, University of California at San Francisco, © 1994); QUANTA/CHARMM (Molecular Simulations, Inc., Burlington, Mass., 1994); or Insight II/Discover (Biosysm Technologies Inc., San Diego, Calif., © 1994).

In another embodiment, once an agent binding to a NS2 protein, or a C-terminal fragment thereof, has been optimally selected or designed, as described above, substitutions may then be made in some of its atoms or side groups in order to improve or modify its binding properties. In one embodiment, initial substitutions are conservative, i.e., the replacement group will have approximately the same size, shape, hydrophobicity and charge as the original group. Such substituted chemical compounds may then be, in another embodiment, analyzed for efficiency of fit to the NS2 protein, or a C-terminal fragment thereof by the same computer methods described in detail, above.

In one embodiment, the agent is an antibody.

In one embodiment, the term “antibody” refers to intact molecules as well as functional fragments thereof, such as Fab, F(ab′)₂, and Fv. In one embodiment, the term “Fab” refers to a fragment, which contains a monovalent antigen-binding fragment of an antibody molecule, and in one embodiment, can be produced by digestion of whole antibody with the enzyme papain to yield an intact light chain and a portion of one heavy chain, or in another embodiment can be obtained by treating whole antibody with pepsin, followed by reduction, to yield an intact light chain and a portion of the heavy chain. In one embodiment, the term “F(ab′)₂, refers to the fragment of the antibody that can be obtained by treating whole antibody with the enzyme pepsin without subsequent reduction, F(ab′)2 is a dimer of two Fab′ fragments held together by two disulfide bonds. In another embodiment, the term “Fv” refers to a genetically engineered fragment containing the variable region of the light chain and the variable region of the heavy chain expressed as two chains, and in another embodiment, the term “single chain antibody” or “SCA” refers to a genetically engineered molecule containing the variable region of the light chain and the variable region of the heavy chain, linked by a suitable polypeptide linker as a genetically fused single chain molecule.

Methods of producing these fragments are known in the art. (See for example, Harlow and Lane, Antibodies: A Laboratory Manual, Cold Spring Harbor Laboratory, New York, 1988, incorporated herein by reference).

Antibody fragments for use according to the methods of the present invention can be prepared, in one embodiment, through proteolytic hydrolysis of an appropriate antibody, or, in other embodiments, by expression in E. coli or mammalian cells (e.g. Chinese hamster ovary cell culture or other protein expression systems) of DNA encoding the fragment.

In some embodiments, antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. In other embodiments, enzymatic cleavage using pepsin can be used to produce two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, in some embodiments, as described in Inbar et al., Proc. Nat'l Acad. Sci. USA 69:2659-62, 1972. In other embodiments, the variable chains can be linked by an intermolecular disulfide bond or cross-linked by chemicals such as glutaraldehyde. In some embodiments, the Fv fragments comprise V_{H} and V_{L} chains connected by a peptide linker. These single-chain antigen binding proteins (sFv) may be prepared by constructing a structural gene comprising DNA sequences encoding the VH and VL domains connected by an oligonucleotide. The structural gene may be inserted into an expression vector, which is subsequently introduced into a host cell such as E. coli. The recombinant host cells may synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which are hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

In another embodiment, the agent is a small molecule. In one embodiment, the phrase “small molecule” refers to, inter-alia, synthetic organic structures typical of pharmaceuticals, peptides, nucleic acids, peptide nucleic acids, carbohydrates, lipids, and others, as will be appreciated by one skilled in the art. In another embodiment, small molecules, may refer to chemically synthesized peptidomimetics of the 6-mer to 9-mer peptides of the invention.

It is to be understood that any compound, such as a crystal, protein or peptide comprising, or derived from a C-terminal domain of NS2, for any use in this invention may be isolated, generated synthetically, obtained via translation of sequences subjected to any mutagenesis technique, or obtained via protein evolution techniques, well known to those skilled in the art, each of which represents an embodiment of this invention, and may be used in the methods of this invention, as well.

In other embodiments, the crystal or peptide comprising, or derived from an C-terminal domain of NS2 of the present invention may be employed in the following applications: 1) screening assays; 2) predictive medicine (e.g., diagnostic assays, prognostic assays, monitoring clinical trials, and pharmacogenetics); and 3) methods of treatment (e.g., therapeutic and prophylactic).

In one embodiment, this invention provides a method for identifying a test compound that inhibits or prevents hepatitis C viral infection or pathogenesis, the method comprising:
(a) contacting a cell in culture harboring replicating hepatitis C virus RNA or infected with a hepatitis C virus in culture with a test compound, under conditions and for a time sufficient to permit the dimerization of NS2 proteins of said virus;
(b) culturing a cell harboring replicating hepatitis C virus RNA or infected with hepatitis virus C virus in the absence of said agent, under conditions and for a time sufficient to permit the dimerization of said NS2 protein; and
(c) comparing viral infection or pathogenic effects on cells cultured in (a) versus (b),
   whereby a decrease or absence of viral infection or pathogenic effects on cells detected in (a) as compared to (b) indicates that the test compound inhibits or prevents hepatitis C viral infection or pathogenesis.

In one embodiment, the compound identified in the screening methods as described, may be identified by computer modeling techniques, and others, as described hereinabove. Verification of the activity of these compounds may be accomplished by the methods described herein, where, in one embodiment, the test compound demonstrably affects HCV infection, replication and/or pathogenesis in an assay, as described. In one embodiment, the assay is a cell-based assay, which, in one embodiment, makes use of primary isolates, or in another embodiment, cell lines, etc. In one embodiment, the cell is within a homogenate, or in another embodiment, a tissue slice, or in another embodiment, an organ culture. In one embodiment, the cell or tissue is hepatic in origin, or is a derivative thereof. In another embodiment, the cell is a commonly used mammalian cell line, which has been engineered to express key molecules known to be, or in another embodiment, thought to be involved in HCV infection, replication and/or pathogenesis.

In another embodiment, this invention provides a method for identifying a test compound that interferes with the autoproteolytic cleavage mediated by the C-terminal domain of an NS2 protein, the method comprising:
a. providing in vitro conditions wherein an NS2/NS3/NS4A protein, or fragment thereof comprising junctional sequences between NS2, NS3 and NS4 is produced, such that said junctional sequences are intact;
b. contacting said NS2/NS3/NS4A protein, or fragment thereof with a test compound, under conditions and for a time sufficient for autoproteolytic cleavage of a junction between NS2 and NS3 of said protein or fragment thereof to occur;
c. contacting said NS2/NS3/NS4A protein, or fragment thereof without said test compound, under conditions and for a time sufficient for autoproteolytic cleavage of a junction between NS2 and NS3 of said protein or fragment thereof to occur;
d. detecting whether said junctional sequences in (b) versus (c) are intact,
   whereby a decrease or absence in intact junctional sequences as detected in (c) as compared to (b) indicates that the test compound interferes with the autoproteolytic activity of a C-terminal domain of an NS2 protein.

In one embodiment, the active site that mediates autoproteolytic cleavage comprises a histidine residue at position 143, a glutamate residue at position 163, a cysteine residue at position 184, a carboxyterminal leucine residue at position 217, or combinations thereof.

In one embodiment, the method is accomplished in a cell-based assay as described. In one embodiment, the cell is infected with an HCV virus, or, in another embodiment, the cell is infected with a recombinant HCV virus, or in another embodiment, the cell comprises a vector which express an NS2/NS31NS4A protein, or fragment thereof comprising junctional sequences between NS2, NS3 and NS4, wherein the protein or fragment is produced in the cell. In one embodiment, the protein, or protein fragment, is engineered to express a detectable marker upon autocleavage of the NS2/NAS3 junction.

In another embodiment, the assay is an in vitro assay, which detects enzymatic activity, and does not require the use of cells.

In one embodiment, the test compound identified by the methods of this invention, as inhibiting HCV infection, or in another embodiment, replication, or in another embodiment, pathogenesis, or in another embodiment, a combination thereof, may effect this activity via its prevention, diminution or abrogation of autoproteolytic activity of the HCV NS2 protein, or in another embodiment, dimerization, as described, or in another embodiment, membrane association, or in another embodiment, a combination thereof. Such a compound thus identified may be used in other embodiments as part of the methods of this invention, for inhibiting HCV infection, or in another embodiment, replication, or in another embodiment, pathogenesis. In other embodiments, the test compounds thus identified, compositions comprising the test compounds, crystallized forms, etc., are to be considered as part of this invention and embodiments thereof. Optimized forms of such compounds may be generated, by methods as will be appreciated by those of skill in the art, which may enhance interactions of key positions within the test compounds and residues within HCV NS2 proteins, and represent embodiments of this invention.

In another embodiment, this invention provides a method for inhibiting hepatitis C viral infection or pathogenesis, comprising contacting an C-terminal domain of an NS2 protein, or a fragment thereof, of hepatitis C virus with an agent that inhibits or suppresses the autoproteolytic activity of NS2.

In one embodiment, according to this aspect of the invention, the agent interferes with attractive forces between histidine 143 and glutamate 163 of a first monomer, and cysteine 184 and leucine 217 of a second monomer.

Active sites of a dimer were shown hereinbelow to be composed of amino acids from both monomers: histidine 143 and glutamate 163, which originate from one NS2 molecule, and cysteine 184 and carboxy-terminal leucine 217, which originated from a second chain. In one embodiment, dimer formation is required for establishing the active sites and thus the enzymatic activity of NS2.

In one embodiment of this invention, the carboxy-terminus of each NS2 molecule remains bound in the corresponding active site after autocleavage, and according to this aspect of the invention, the active site is inaccessible for other substrates. In one embodiment, the consequence of such a proposed mechanism is that each NS2 protease subunit can cleave only once, representing a means of the virus to tightly coordinate proteolytic processing at the NS2/3 junction, which in some aspects of this invention, relate to HCV pathogenesis.

According to this aspect of the invention, and in one embodiment, the agent prevents adherence of the C-terminal domain of an NS2 protein to the active site, following protein cleavage. In one embodiment, the agent prevents tight regulation of cleavage of the NS2/NS3, resulting in enhanced cleavage of other substrates, including, in some embodiments, other junctional regions, or in another embodiments, other sites within the polyprotein.

In another embodiment, this invention provides a method for inhibiting hepatitis C viral infection or pathogenesis, comprising contacting an NS2 protein, or a fragment thereof, of hepatitis C virus with an agent that inhibits or suppresses dimerization of said NS2 protein.

In another embodiment, any method of inhibiting hepatitis C viral infection or pathogenesis may further comprise the administration of an interferon, such as, for example, Intron-A (interferon alpha-2b) by Schering, PEG-INTRON (pegylated interferon alpha-2b) by Schering, Roferon-A (interferon alfa-2a) by Roche, PEGASYS (pegylated interferon alfa-2a) by Roche, INFERGEN (interferon alfacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and Interferon gamma-1b by InterMune.

In another embodiment, any method of this invention which inhibits hepatitis C viral infection and/or replication and/or pathogenesis may further comprise the administration of nucleoside analogs, such as, for example, synthetic guanosine analogs, such as, for example, ribavirin.

In another embodiment, the method of inhibiting hepatitis C viral infection or pathogenesis may comprise the administration of a crystallized C-terminal domain of an NS2 protein, or a mutated version thereof, wherein the protein serves to interfere with dimer formation, in one embodiment, such that the fragment dimerizes with an HCV NS2 protein expressed in an infected cell, such that functional dimer formation is prevented. In one embodiment, the mutated NS2 protein results in improper formation of the active site, or in another embodiment, prevents dimerization, such that the NS2 protein cannot exert its enzymatic function, thereby inhibiting hepatitis C viral infection or pathogenesis.

In another embodiment, the structure, and in another embodiment, function of NS2 proteins, and in another embodiment, C-terminal domains thereof, is conserved among other Flaviviridae. According to this aspect of the invention, and in another embodiment, methods of this invention utilizing the C-terminal domain of HCV NS2 protein are applicable to other Flaviviridae. In one embodiment, screening methods utilizing HCV C-terminal domains of NS2 protein may be utilized for identifying inhibitors of other Flaviviridae, or in another embodiment, C-terminal domains of NS2 protein of the respective Flaviviridae may be used.

In one embodiment, the C-terminal domains of Flaviviridae NS2 protein have a sequence such as that disclosed in Genbank Accession Number: AAB66324, NP_{—}777540, NP_{—}777501, NP_{—}777488, NP_{—}776266, NP_{—}803204, NP_{—}777514, NP_{—}757356, AAL25622, CAC83235, or one homologous thereto.

In one embodiment, the terms “homology”, “homologue” or “homologous”, refer to a molecule, which exhibits, in one embodiment at least 70% correspondence with the indicated molecule, in terms of, in one embodiment, its structure, or in another embodiment, amino acid sequence. In another embodiment, the molecule exhibits at least 72% correspondence with the indicated sequence or structure. In another embodiment, the molecule exhibits at least 75% correspondence with the indicated sequence or structure. In another embodiment, the molecule exhibits at least 80% correspondence with the indicated sequence or structure. In another embodiment, the molecule exhibits at least 82% correspondence with the indicated sequence or structure. In another embodiment, the molecule exhibits at least 85% correspondence with the indicated sequence or structure. In another embodiment, the molecule exhibits at least 87% correspondence with the indicated sequence or structure. In another embodiment, the molecule exhibits at least 90% correspondence with the indicated sequence or structure. In another embodiment, the molecule exhibits at least 92% correspondence with the indicated sequence or structure. In another embodiment, the molecule exhibits at least 95% or more correspondence with the indicated sequence or structure. In another embodiment, the molecule exhibits at least 97% correspondence with the indicated sequence or structure. In another embodiment, the molecule exhibits at least 99% correspondence with the indicated sequence or structure. In another embodiment, the molecule exhibits 95%-100% correspondence with the indicated sequence or structure. Similarly, as used herein, the reference to a correspondence to a particular molecule includes both direct correspondence, as well as homology to that molecule as herein defined.

Homology, as used herein, may refer to sequence identity, or may refer to structural identity, or functional identity. By using the term “homology” and other like forms, it is to be understood that any molecule, that functions similarly, and/or contains sequence identity, and/or is conserved structurally so that it approximates the reference molecule, is to be considered as part of this invention.

In one embodiment, determining inhibition of HCV replication and/or infection and/or pathogenesis may be accomplished via incubating the test compound, or in another embodiment, the agent, or in another embodiment, a crystal of this invention, in a medium with a liver slice prepared from a subject having hepatitis for a period of time, e.g., 24 to 96 hours, and then determining the replication level of the virus, such as genome level, protein level, or the replication rate of the virus, in the liver slice. One may also determine a control replication level of the virus in a second liver slice in the same manner except that the second liver slice is incubated in a medium free of the compound. If the replication level in the first slice is lower than that in the second slice, the compound is to be considered as inhibiting HCV replication and/or infection and/or pathogenesis.

A liver slice can be prepared using techniques well known in the art. It can be prepared in different dimensions and maintained in various culture systems. See, e.g., Groneberg et al., Toxicol. Pathol. 30 (2002) 394-399 and Ekins Drug Metab. Rev. 28 (1996) 591-623. A plurality of liver slices can be obtained from a subject and stored in, e.g., liquid nitrogen, for later use (Isacheako et al., Eur. J. Obstet. Gynecol. Reprod. Biol. 2003 Jun. 10; 108(2):186-93). These slices can also be used in parallel to screen different compounds, thereby achieving high-throughput screening.

The replication level of a virus can be determined, in other embodiments, using techniques known in the art. For example, the genome level can be determined using RT-PCR. To determine the level of a viral protein, one can use techniques including ELISA, immunoprecipitation, immunofluorescence, EIA, RIA, and Western blotting analysis. To determine the replication rate of a virus, one can use the method described in, e.g., Billaus et al., Virology 26 (2000) 180-188.

A viral replicon is a subgenomic viral replication system, derived from a viral genome, that is capable of replicating within cells cultured in vitro (Agapov et al., 1998). They typically encode all of the cis- and trans-acting viral components required for replication and transcription of the viral genome within a cell, but lack one or more functional element required for full virus replication. The element could be lacking due to a deletion of all or part of the sequence encoding that function, or the element could be lacking due to a mutation, such as a point mutation, rendering the element nonfunctional. Recently several reports have described the selection of replicons capable of persistent replication in cells.

Cell cultures comprising replicons offer a number of benefits in discovery and analysis of antiviral agents. They permit the effect of an antiviral agent to be observed in the context of living cells, so that any agents that show antiviral activity necessarily enter and act within living cells. Replicon-containing cell cultures also allow the immediate identification of antiviral agents with obvious undesirable cytotoxicity using well established cytotoxicity assays. These cell cultures also permit cell-based drug discovery screens and other studies to be performed against viruses such as hepatitis C virus (HCV) and human papillomavirus (HPV) that are unable to be conventionally cultured in vitro. Since viral functions related to infectivity are typically not required for viral genome replication, viral replicons lacking at least one infectivity-related sequence are much safer and thus easier to work with than infectious virus.

In one embodiment, the screening methods of this invention may be conducted as described or modified from that described in U.S. Pat. Nos. 6,750,009; 6,689,559; 6,630,343; 6,777,395, and/or may employ constructs described therein.

In another embodiment, determining inhibition of HCV replication and/or infection and/or pathogenesis may be accomplished via determining a responsiveness of a subject to an agent, or in another embodiment, a test compound identified via the methods of this invention, or in another embodiment, a crystal of this invention.

To evaluate a subject's responsiveness to such materials, in one embodiment, a number of liver slices from the subject are prepared, and the slices are incubated with the materials, respectively. A replication level of the virus in each of the liver slices is determined, and compared to a control level in the manner described above, in one embodiment. The subject is determined to be responsive to the material if the replication level in a slice incubated with the material is lower than the control level.

This method can be used, in other embodiments, as a means of monitoring hepatitis treatment in a subject. For this purpose, liver slices may be prepared from a subject before, during, and after undergoing treatment. The slices are then subjected to the treatment in vitro, and the replication level of the virus in each slice is obtained in the manner described above.

In another embodiment, the inhibition of HCV replication and/or infection and/or pathogenesis includes inhibition of downstream effects of HCV or infection with other Flaviviridae. In one embodiment, downstream effects include neoplastic disease, including, in one embodiment, the development of hepatocellular carcinoma.

In one embodiment, the molecular architecture of the dimerized C-terminal domains of the NS2 supports, and in another embodiment, maximizes protein-protein interactions with other proteins.

In another embodiment, protein, or in another embodiment, peptide or in another embodiment, other inhibitors of the present invention cause inhibition of infection, replication, or pathogenesis of hepatitis C Virus in vitro or, in another embodiment, in vivo when introduced into a host cell containing the virus, and may exhibit, in another embodiment, an IC50 in the range of from about 0.0001 nM to 100 μM in an in vitro assay for at least one step in infection, replication, or pathogenesis of HCV, more preferably from about 0.0001 nM to 75 ?M, more preferably from about 0.0001 nM to 50 ?M, more preferably from about 0.0001 nM to 25 ?M, more preferably from about 0.0001 nM to 10 ?M, and even more preferably from about 0.0001 nM to 1 ?M.

In another embodiment, the inhibitors of HCV infection, or in another embodiment, replication, or in another embodiment, pathogenesis, may be used, in another embodiment, in ex vivo scenarios, such as, for example, in routine treatment of blood products wherein a possibility of HCV infection exists, when serology indicates a lack of HCV infection.

In another embodiment, this invention provides for compositions comprising a crystallized C-terminal domain of an NS2 protein, or a mutant thereof, or a homologue thereof, wherein the homologue exhibits significant structural or sequence homology to the NS2 protein. In another embodiment, this invention provides for compositions comprising an agent, as herein described, which inhibits hepatitis C viral infection or pathogenesis, obtained via the methods of this invention.

In one embodiment, the term “composition” refers to any such composition suitable for administration to a subject, and such compositions may comprise a pharmaceutically acceptable carrier or dilutant, for any of the indications or modes of administration as described. The active materials in the compositions of this invention can be administered by any appropriate route, for example, orally, parenterally, intravenously, intradermally, subcutaneously, or topically, in liquid or solid form.

In one embodiment, the dose of the active ingredient is in the range from about 1 to 50 mg per kg of body weight per day, or in another embodiment, 1 to 20 mg per kg of body weight per day, or in another embodiment, 0.1 to about 100 mg per kg of body weight per day. The effective dosage range of the active ingredient/s can be calculated by means known to those skilled in the art.

The active ingredient in the compositions of this invention can be conveniently administered in any suitable unit dosage form, including but not limited to one containing 7 to 3000 mg, preferably 70 to 1400 mg, of active ingredient per unit dosage form. For example, an oral dosage of 50-1000 mg of the active ingredient may be convenient.

In one embodiment, the active ingredient is administered to achieve peak plasma concentrations of from about 0.2 to 70 ?M, or in another embodiment, from about 1.0 to 10 ? M. This can be achieved, for example, by the intravenous injection of a 0.1 to 5% solution of the active ingredient, optionally in saline, or administered as a bolus of the active ingredient.

The concentration of active compound in the drag composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

In one embodiment, the mode of administration of the active compound is oral. Oral compositions may, in another embodiment, include an inert diluent or an edible carrier. They can be enclosed in gelatin capsules or compressed into tablets. In one embodiment, the active compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The active ingredient can be administered as a component of an elixir, suspension, syrup, wafer, chewing gum or the like. A syrup can contain, in addition to the active compounds, sucrose as a sweetening agent and certain preservatives, dyes and colorings and flavors.

The active ingredient may, in other embodiments, be mixed with other active materials that do not impair the desired action, or with materials that supplement the desired action, such as antibiotics, antifungals, anti-inflammatories, or other antivirals, including nucleoside inhibitors. Solutions or suspensions used for parenteral, intradermal, subcutaneous, or topical application can include, in other embodiments, the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

In another embodiment, compositions for intravenous administration may comprise carriers such as physiological saline or phosphate buffered saline (PBS).

In another embodiment, the active ingredients may be prepared with carriers, which protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antibodies to viral antigens) represent other embodiments of pharmaceutically acceptable carriers. These can be prepared according to methods known to those skilled in the art, for example, as described in U.S. Pat. No. 4,522,811, which is incorporated herein by reference in its entirety. For example, liposome formulations can be prepared by dissolving appropriate lipid(s), such as stearoyl phosphatidyl ethanolamine, stearoyl phosphatidyl choline, arachadoyl phosphatidyl choline, and/or cholesterol, in an inorganic solvent that is then evaporated, leaving behind a thin film of dried lipid on the surface of the container. An aqueous solution of the active ingredient is then introduced into the container. The container is swirled by hand to free lipid material from the sides of the container and to disperse lipid aggregates, thereby forming the liposomal suspension.

The active ingredient(s) are included in the pharmaceutically acceptable carrier or diluent in an amount sufficient to deliver to a subject a therapeutically effective amount of compound to inhibit viral replication, or infection, in vivo, especially Flaviviridae replication, without causing serious toxic effects in the treated patient. By “inhibitory amount” is meant an amount of active ingredient sufficient to exert an inhibitory effect as measured by, for example, an assay such as the ones described herein.

In one embodiment, the composition may be formulated to provide controlled delivery. In one embodiment, such a composition may comprise a biodegradable polymer such as polylactic acid (Kulkarni et al., in 1966 “Polylactic acid for surgical implants,” Arch. Surg., 93:839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers). See also U.S. Pat. No. 5,626,863 to Hubbell, et al which describes photopolymerizable biodegradable hydrogels as tissue contacting materials and controlled release carriers (hydrogels of polymerized and crosslinked macromers comprising hydrophilic oligomers having biodegradable monomeric or oligomeric extensions, which are end capped monomers or oligomers capable of polymerization and crosslinking); and WO 97/05185 to Focal, Inc. directed to multiblock biodegradable hydrogels for use as controlled release agents for drug delivery and tissue treatment agents.

Degradable materials of biological origin, such as crosslinked gelatin, are well known, and represent other embodiments for use in compositions of this invention. Another example is hyaluronic acid, which has been crosslinked and used as a degradable swelling polymer for biomedical applications (U.S. Pat. No. 4,957,744 to Della Valle et. al.; (1991) “Surface modification of polymeric biomaterials for reduced thrombogenicity,” Polym. Mater. Sci. Eng., 62:731-735]).

Many dispersion systems are currently in use, or being explored for use, as carriers of substances, and particularly of biologically active compounds. Dispersion systems used for pharmaceutical and cosmetic formulations can be categorized as either suspensions or emulsions. Suspensions are defined as solid particles ranging in size from a few manometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses yet another method for drug delivery by forming an implant in situ by dissolving a nonreactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

It is to be understood that any applicable drug delivery system may be used with the compositions and/or agents/crystals of this invention, for administration to a subject, and is to be considered as part of this invention.

For example, U.S. Pat. No. 5,578,325 discloses the use of nanoparticles and microparticles of non-linear hydrophilic hydrophobic multiblock copolymers for drug delivery. U.S. Pat. No. 5,545,409 discloses a delivery system for the controlled release of bioactive factors. U.S. Pat. No. 5,494,682 discloses the use of ionically cross-linked polymeric microcapsules as a drug delivery system. U.S. Pat. No. 5,728,402 to Andrx Pharmaceuticals, Inc. describes a controlled release formulation that includes an internal phase that comprises the active drug, its salt or prodrug, in admixture with a hydrogel forming agent, and an external phase which comprises a coating that resists dissolution in the stomach. U.S. Pat. Nos. 5,736,159 and 5,558,879 to Andrx Pharmaceuticals, Inc. disclose controlled release formulations for drugs with little water solubility in which a passageway is formed in situ. U.S. Pat. No. 5,567,441 to Andrx Pharmaceuticals, Inc. discloses a once-a-day controlled release formulation. U.S. Pat. No. 5,508,040 discloses a multiparticulate pulsatile drug delivery system. U.S. Pat. No. 5,472,708 discloses a pulsatile particle based drug delivery system. U.S. Pat. No. 5,458,888 describes a controlled release tablet formulation which can be made using a blend having an internal drug containing phase and an external phase which comprises a polyethylene glycol polymer which has a weight average molecular weight of from 3,000 to 10,000. U.S. Pat. No. 5,419,917 discloses methods for the modification of the rate of release of a drug form a hydrogel which is based on the use of an effective amount of a pharmaceutically acceptable ionizable compound that is capable of providing a substantially zero-order release rate of drug from the hydrogel. U.S. Pat. No. 5,458,888 discloses a controlled release tablet formulation.

U.S. Pat. No. 5,641,745 to Elan Corporation, plc discloses a controlled release pharmaceutical formulation which comprises the active drug in a biodegradable polymer to form microspheres or nanospheres. The biodegradable polymer is suitably poly-D,L-lactide or a blend of poly-D,L-lactide and poly-D,L-lactide-co-glycolide. U.S. Pat. No. 5,616,345 to Elan Corporation plc describes a controlled absorption formulation for once a day administration that includes the active compound in association with an organic acid, and a multi-layer membrane surrounding the core and containing a major proportion of a pharmaceutically acceptable film-forming, water insoluble synthetic polymer and a minor proportion of a pharmaceutically acceptable film-forming water soluble synthetic polymer. U.S. Pat. No. 5,641,515 discloses a controlled release formulation based on biodegradable nanoparticles. U.S. Pat. No. 5,637,320 discloses a controlled absorption formulation for once a day administration. U.S. Pat. Nos. 5,580,580 and 5,540,938 are directed to formulations and their use in the treatment of neurological diseases. U.S. Pat. No. 5,533,995 is directed to a passive transdermal device with controlled drug delivery. U.S. Pat. No. 5,505,962 describes a controlled release pharmaceutical formulation.

The compositions of the invention can be administered as conventional HCV therapeutics. The compositions of the invention may include more than one active ingredient which interrupts or otherwise alters the active site, in one embodiment, or dimerization, in another embodiment, or membrane association, in another embodiment.

The precise formulations and modes of administration of the anti-HCV compounds, or in another embodiment, compositions of the invention will depend on the nature of the anti-HCV agent, the condition of the subject, and the judgment of the practitioner. Design of such administration and formulation is routine optimization generally carried out without difficulty by the practitioner.

The following examples are intended to illustrate but not limit the present invention.

### EXAMPLES

### Materials and Experimental Methods

### Cloning

A fragment encompassing amino acids 94 to 217 of the NS2 coding sequence from HCV genotype 1a strain H77 (corresponding to residues 903 to 1026, numbered according to the HCV polyprotein sequence) was amplified by PCR. An NdeI restriction site was inserted at the 5′ end, and a stop codon immediately downstream of the NS2 coding sequence followed by an XhoI restriction site were included at the 3′ end. This fragment was cloned into the pET28a expression vector (Novagen), which contains a 5′ cassette containing an AUG initiation codon, followed by the coding sequence for a hexahistidine tag, and a consensus coding sequence for cleavage by thrombin upstream of the cloning site. Proper amplification and insertion were tested by sequencing of the coding region of the pET28a NS2 903-1026 vector.

### Protein Expression and Purification

For the expression of NS2 903-1026, 1 liter of LB medium supplemented with 30 μg kanamycin per liter were inoculated from an overnight culture of Escherichia coli BL21(DE3) transformed with the pET28a NS2 903-1026 plasmid such that the cell density was at approximately OD (600 nm) of 0.05. Cells were grown at 30° C. and 250 rpm until an OD (600 nm) of 1.0-1.1 was reached. The bacteria were then chilled at 4° C. for 30 min, induced with 1 mM isopropyl-1-thio-?-D-galactopyranoside (IPTG), and incubated at 18° C. and 250 rpm for 20 h. Cells were then collected by centrifugation at 4,000×g for 15 min and resuspended in 15 ml lysis buffer (25 mM Tris-HCl pH 7.5, 150 mM KCl, 10% glycerol, and 3% Triton X-100) per liter. Cells were lysed by three passes through a cold Avestin air emulsifier at 10,000 to 15,000 psi. Following lysis, cell extracts were clarified at 30,000×g for 30 min at 4° C. Imidazol was added to the clarified extracts to a final concentration of 30 mM. The extracts were then loaded on a 5-ml bed volume HiTrap nickel immobilized metal affinity chromatography (IMAC) column (Amersham Biosciences) equilibrated with buffer 1 (25 mM Tris-HCl pH 7.5, 150 mM KCl, 10% glycerol, 3% Triton X-100, and 30 mM imidazol) at a flow rate of 1 ml/min. Following extensive washing with this buffer, detergent was exchanged by a linear gradient from buffer 1 to buffer 2 (50 mM Tris-HCl pH 7, 150 mM KCl, 10% glycerol, 1% n-octyl-?-glucopyranoside [nOG], and 30 mM imidazol) at 1 ml/min for 10 min. After washing with buffer 2, NS2 was eluted with buffer 2 supplemented with 300 mM imidazol. Fractions containing NS2 were pooled, followed by removal of the amino-terminal hexahistidine tag by cleavage with thrombin at 4° C. overnight. Subsequently, the protein was further purified by cation exchange chromatography on a 1 ml bed volume HiTrap SP column (Amersham/Pharmacia) equilibrated with 10 ml buffer 3 (50 mM MES pH 6, 10% Glycerol, 1% nOG) at 1 ml/min. To decrease the salt concentration and lower the pH, a two-fold excess volume of buffer 3 was added to the NS2 pool prior to loading at 1 ml/min. After washing with buffer 3, the protein was eluted with a linear 1 M KCl gradient at 1 ml/min. Fractions containing NS2 were pooled and loaded onto a Sephacryl S-200 gel filtration column (Amersham/Pharmacia) equilibrated with buffer 4 (50 mM MES pH 6, 150 mM KCl, 10% glycerol, 5.4 mM n-decyl-?-maltopyranoside [DM]) at 0.8 ml/min. The eluted protein was concentrated by cation exchange chromatography on a 1 ml bed volume Resource S column (Amersham/Pharmacia) equilibrated with buffer 5 (50 mM MES pH 6, 10% glycerol, 5.4 mM DM). An equal volume of buffer 5 was added to the protein prior to loading at 1 ml/min. Following washing with buffer 5, the protein was eluted with buffer 5 supplemented with 350 mM KCl at 1.5 ml/min. Concentration of purified NS2 was determined using the Bio-Rad protein assay. Final protein yields were typically between 1 and 2 mg per liter of bacterial expression culture. The purity of the protein assessed by SDS-PAGE was greater than 95%.

### Expression and Purification of Selenomethionine Labeled Protein

For expression of selenomethionine-labeled NS2, Escherichia coli BL21(DE3) were grown in minimal medium supplemented with L-amino acids except methionine, and 50 mg/ml selenomethionine (Doublie, S. Methods Enzymol. 276, 523-530 (1997)). Bacteria were inoculated and grown as described for the non-labeled protein. Cells were induced with IPTG at an OD (600 nm) of 0.8-0.9. Following expression, NS2 was purified as described above. Protein yields were between 1 and 2 mg per liter of bacterial culture, which is in the same range as for non-labeled protein.

### Crystal Growth and Freezing

Crystals of NS2 were grown by hanging drop vapor diffusion at 4?C on siliconized cover slips in 24 well Linbro plates. The 500 microliters well solution contained 100 mM Tris pH 8.5, 0.8 M ammonium acetate, 0.25 M lithium chloride and 12% (w/v) polyethylene glycol 3350. The drop consisted of 2 microliters of NS2 903 at 6 to 9 mg/ml in 50 mM MES pH 6, 350 mM KCl, 10% glycerol, 5.4 mM DM, and 2 microliters of well solution. Cubic or rhombic crystals of 0.1 to 0.2 mm in size grew from these conditions in approximately 6 days. For freezing, crystals were transferred from hanging drops to well solution supplemented with 25% glycerol and 5.4 mM DM at 4?C over the course of 45 min. Subsequently, crystals were harvested and flash frozen in liquid propane.

### Data Collection

The selenomethionine data sets were collected at beamline X9A, National Synchrotron Light Source, Brookhaven National Labs, Upton, N.Y. MAD data were collected at two wavelengths corresponding to the peak (?_{λ}10.97927 Å) and inflection point (?2, 0.97939 Å) of the selenium K absorption edge (Table 1). A native data set at 2.3 Å resolution was collected at beamline X29, National Synchrotron Light Source, at a wavelength of 1.1 Å.

### Data Processing and Model Building

Data obtained from the selenium wavelengths were processed and scaled from 30 to 2.9 Å resolution using DENZO/SCALEPACK (Otwinowski, Z. & Minor, M. in Macromolecular Crystallography, part A, 307-326 (Academic Press (New York), 1997)). The space group was P2₁, with unit cell dimensions a=61.23 Å, b=67.27 Å, c=108.87 Å, and ?=105.82°, with six NS2 molecules per asymmetric unit. Using the anomalous signal from the peak selenomethionine data set, 19 of the 24 selenium sites were found using SnB (Weeks, C. M. & Miller, R., The design and implementation of SnB v2.0, J. Appl. Cryst. 32, 120-124 (1999)). An interpretable electron density map was obtained using MLPHARE followed by density modification and phase combination by SOLOMON and DM (Collaborative Computational Project, N. The CCP4 suite: programs for protein crystallography. Acta Crystallogr D Biol Crystallogr 50, 760-3 (1994)). Several rounds of iterative model building and refinement were performed using the programs O (Jones, T. A., Zou, J. Y., Cowan, S. W. & Kjeldgaard. Improved methods for building protein models in electron density maps and the location of errors in these models. Acta Crystallogr A 47 (Pt 2), 110-9 (1991)) and CNS (Brunger, A. T. et al. Crystallography & NMR system: A new software suite for macromolecular structure determination. Acta Crystallogr D Biol Crystallogr 54 (Pt 5), 905-21 (1998)).

### Example 1

### NS2 Crystal Characteristics

The native data set was processed and scaled from 30 to 2.28 Å as described above. The space group was P2₁, with unit cell dimensions a=109.81 Å, b=68.82 Å, c=125.16 Å, and ?=105.88°, The asymmetric unit contained twelve NS2 molecules. The native structure was determined by molecular replacement with the program MOLREP (Collaborative Computational Project, N. The CCP4 suite: programs for protein crystallography. Acta Crystallogr D Biol Crystallogr 50, 760-3 (1994)), using a search model containing the partially refined structure from the selenium data sets. The high-resolution model was further refined by several iterative rounds using the programs O and CNS.

A summary of the current refinement statistics is provided in table 1. The current model has an R-factor of 29.2% and free R-factor of 33.9%, with r.m.s.d. on bond lengths and angles of 0.008 Å and 1.583°, respectively. The thermal parameter r.m.s.d. values are 1.09 Å² for main chain atoms and 1.87 Å² for side chain atoms. Graphics were generated using the programs PyMOL (DeLano, W. L. (http;//www.pymol.org, 2002)). GRASP was used for calculating surface potentials (Nicholls, A., et al. Proteins: Structure, Function and Genetics 11, 281-296 (1991)). Sequence alignments were performed using ClustalX (Thompson, J. D., et al. Nucleic Acids Res. 24, 4876-4882 (1997)) and plotting of conservation to molecular surfaces was performed using the program msf_similarity_to_pdb (Dr. David Jeruzalmi, personal communication).

TABLE 1
Summary of data collection and refinement statistics
Data Collection

| | | | | | | | |
|---|---|---|---|---|---|---|---|
| | | | | | | | Phasing |
| Wavelength | | Resolution | Reflections^{a} | Completeness^{a} | R_{sym}^{a,b} | | Power^{c} |
| (Å) | Beamline | (Å) | measured/unique | (%) | (%) | I/? ?I) | (anomalous) |

MAD datasets

| | | | | | | | |
|---|---|---|---|---|---|---|---|
| ??? 1 = 0.97927 | BNL X9A | 30.0-2.90 | 449,457/19,183 | 99.6 (99.3) | 5.4 (25.9) | 20.2 (4.0) | 1.39 |
| ??? [symbol] = 0.97939 | BNL X9A | 30.0-2.95 | 452,880/18,191 | 99.6 (99.5) | 5.6 (26.8) | 20.5 (4.3) | 1.43 |

Native dataset

| | | | | | | |
|---|---|---|---|---|---|---|
| ??? 1 = 1.10000 | BNL X29 | 30.0-2.28 | 535,648/82,381 | 99.6 (96.4) | 8.7 (17.5) | 13.1 (6.1) |

| | | | |
|---|---|---|---|
| Overall MAD figure of merit | acentric | centric | overall |

| | | |
|---|---|---|
| 0.48 | 0.36 | 0.46 |

Refinement against native dataset

| Resolution | | | Completeness | R_{cryst}^{d} | R_{free}^{e} |
|---|---|---|---|---|---|
| (Å) | Cut-off | Reflections | (%) | (%) | (%) |
| 30.0-2.28 | \|F\|/?\|F\| > 2.0 | 75,215 | 91.4 | 29.2 | 33.9 |

root mean square deviations

| | | Thermal parameters | Thermal parameters |
|---|---|---|---|
| Bond lengths | Bond angles | mainchain atoms | sidechain atoms |
| 0.008 Å | 1.583° | 1.09 Å² | 1.87 Å² |

SeMet crystal Space Group P2₁, unit cell a = 61.32 Å, b = 67.41 Å, c = 109.04 Å, ? = 105.83°, 6 molecules per AU
Native crystal Space Group P2₁, unit cell a = 109.81 Å, b = 68.82 Å, c = 125.17 Å, ? = 105.88°. 12 molecules per AU
^{a}Values reported in the format: overall data (last resolution shell)
^{b}R_{sym} = ?|I − <I>|/?I, where I is observed intensity and <I> is average intensity obtained from multiple observations of symmetry-related reflections.
^{c}Phasing power = rms (|F_{H}|/E), where |F_{H}| = heavy atom structure factor amplitude and E = residual lack of closure.
^{d}R_{cryst} = ?|F_{obs} − F_{calc}|/?|F_{obs}|, where F_{obs} and F_{calc} are the observed and calculated structure factors, respectively.
^{e}R_{free} is the same as R_{cryst}, but is calculated with 10% of the data.

### Example 2

### Architecture of NS2

The crystallization condition produced two crystal forms of the C-terminal domain of NS2. Both crystal forms had the same space group (P2₁), ? angle, and two unit cell edges, whereas the third axis was twice as long in one form compared to the other. The smaller unit cell contained a hexamer of NS2 per asymmetric unit, while the larger unit cell was composed of twelve molecules of NS2 per asymmetric unit, arranged as two hexamers. Within the hexamer, NS2 was organized into three tightly packed dimers. All molecules in the asymmetric unit had roughly the same fold.

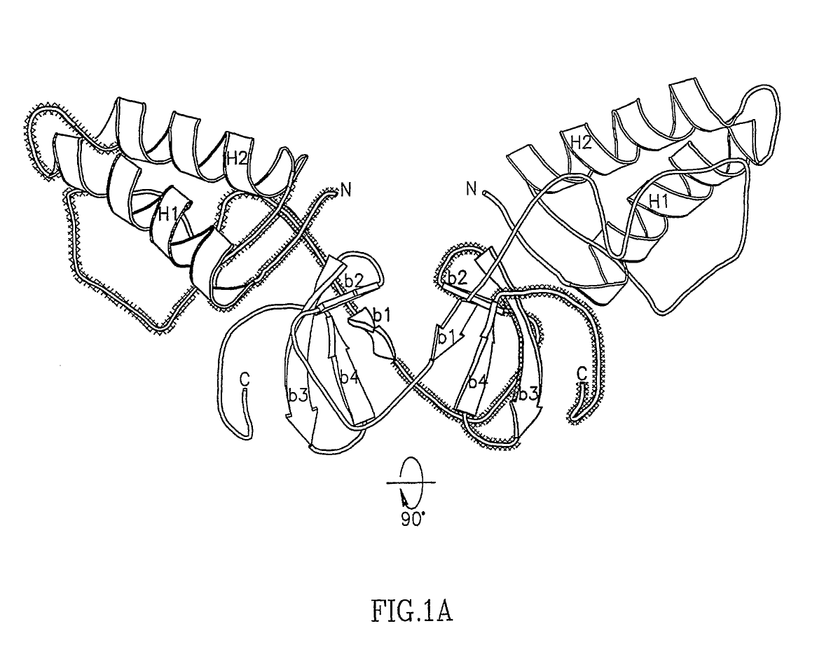

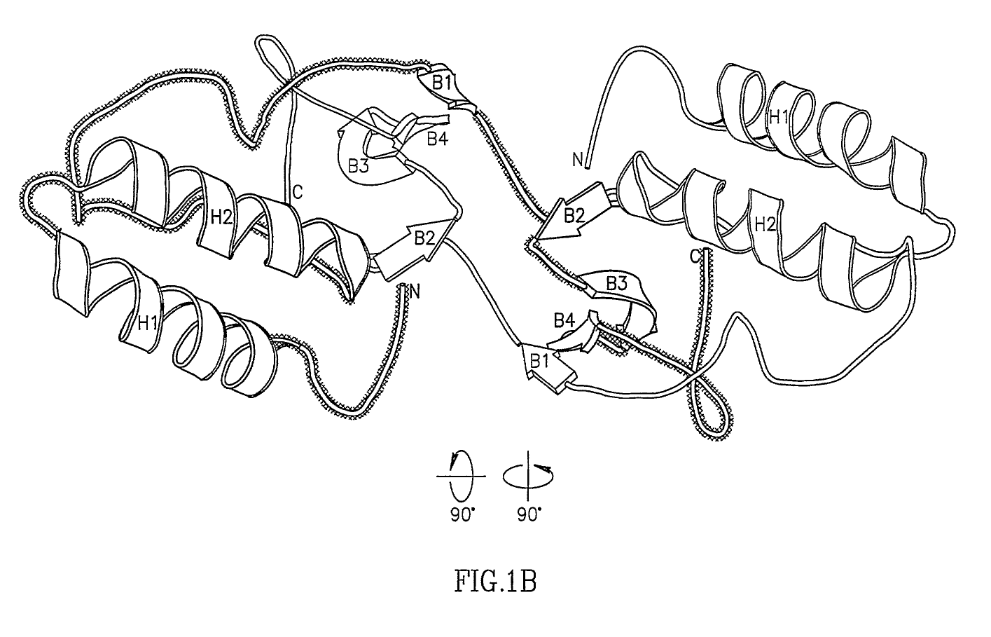

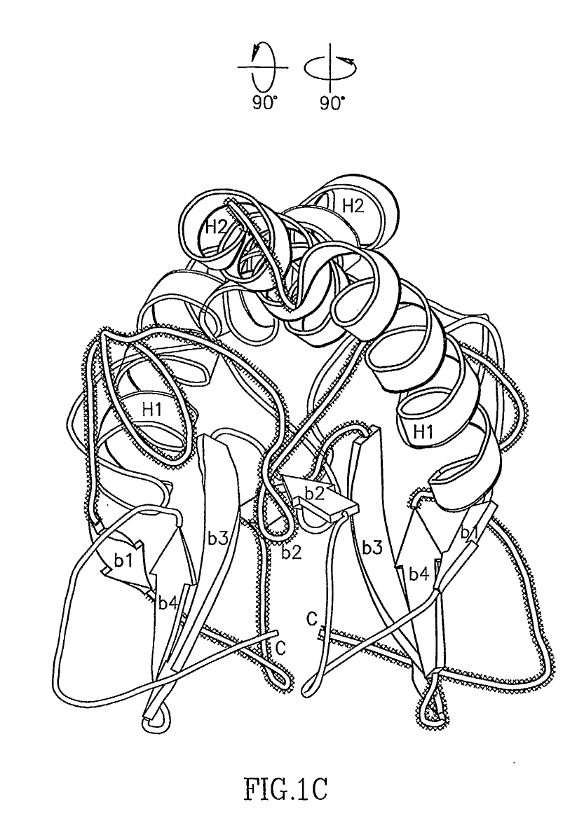

The overall shape of the NS2 dimer resembles a ‘butterfly’, with a two-fold symmetry along the vertical axis in FIG. 1A. The two protein monomers have a very similar overall fold, with an amino-terminal, helical subdomain and a carboxy-terminal subdomain containing an antiparallel beta sheet. Surprisingly, the two molecules of the NS2 dimer contain a cross-over in the middle of the molecule, thereby forming a domain swap (FIGS. 1A and 1B). The amino-terminal subdomain of one molecule in the dimer interacts with the carboxy-terminal subdomain of the other molecule and vice versa. The two subunits of the NS2 dimer make extensive contacts with each other, with a total buried surface area of roughly 1100 Å². The amino-termini of the two monomers lie close to each other, with a distance of around 18 Å between them. The carboxy-termini are positioned on opposite sides of the molecule and are solvent-exposed.

Each NS2 monomer contains two alpha helices (referred to as H1 and H2) and four beta strands (b1 to b4). H1 and H2 are antiparallel helices connected by a short loop in the amino-terminal subdomain of NS2. Following the second helix, the protein has a random-coil conformation that makes contacts with both H1 and H2. The protein then extends into a long linker arm responsible for the cross-over. The arm contains a small beta strand in the middle (b1), which is part of the antiparallel beta sheet in the carboxy-terminal subdomain of the other molecule in the dimer. The protein then makes a relatively sharp turn before entering the antiparallel beta sheet consisting of strands b2, b3, and b4. The carboxy-terminus lies on the side of the beta sheet.

### Example 3

### The NS2 Active Site

The active site of NS2 mediates the autoproteolytic processing at the carboxy-terminus of NS2. The cleavage mechanism is similar to papain-like cysteine proteases, with the catalytic triad consisting of histidine 143, glutamate 163, and cysteine 184 (FIG. 2). Cysteine 184 acts as a nucleophile attacking the carbonyl group at the carboxy-terminal residue of NS2, leucine 217. Histidine 143 provides the proton required for the amine leaving group to generate the amino-terminus of NS3. Histidine 143 is further stabilized by the formation of a hydrogen bond between the carboxyl group of glutamate 163 and one of the secondary amines of histidine 143. Interestingly, the active sites of the dimer are composed of amino acids from both monomers: histidine 143 and glutamate 163 originate from one NS2 molecule, while cysteine 184, as well as the carboxy-terminal leucine 217, are provided by the other chain. This implies that dimer formation is required for establishing the active sites and thus the enzymatic activity of NS2. The carboxy-terminus of each NS2 molecule remains bound in the corresponding active site after cleavage, thereby rendering the active site inaccessible for other substrates. Therefore, each NS2 protease subunit can cleave only once, which may represent a means of the virus to tightly coordinate proteolytic processing at the NS2/3 junction.

### Example 4

### Analysis of the Molecular Surfaces of NS2

‘Front’ and ‘top’ views of the solvent-accessible surface of NS2 colored by electrostatic potential are shown in FIGS. 3A and 3B, respectively. Generally, the molecule has a high content of neutral and basic regions, whereas only a few acidic patches are present in the carboxy-terminal subdomain. The surface on the ‘top’ of the molecule is mainly hydrophobic, with a few basic residues lying underneath. This side may contribute to the association of NS2 to cellular membranes. Acidic regions include the active site, which contains the carboxyl group from the carboxy-terminal residue of the molecule. Other acidic parts are found in the cross-over arm and underneath helices H1 and H2 in the amino-terminal subdomain of the protein.

The green, solvent-accessible surface in FIG. 3C highlights conserved residues of NS2, based on an alignment of NS2 from the 30 HCV genotypes and the related GB virus B reference sequences. The other molecule of the dimer is represented as a ball-and-stick model with the main chain shown as thick ‘worm’. Besides residues surrounding the active site, the surface of NS2 is highly conserved along the dimer interface. The linker arm between the two subdomains is a particularly interesting region, in which the side of the arm that interacts with the other monomer is highly conserved, whereas the solvent-accessible side of the arm shows sequence diversity. Another conserved surface feature can be found at the interface between the amino-terminal subdomain of one molecule and the carboxy-terminal subdomain of the other subunit. The surface in the region of the beta sheet in the carboxy-terminal subdomain is also well conserved and might represent a region involved in interactions with other viral or host factors.

### Example 5

### Membrane Association of NS2

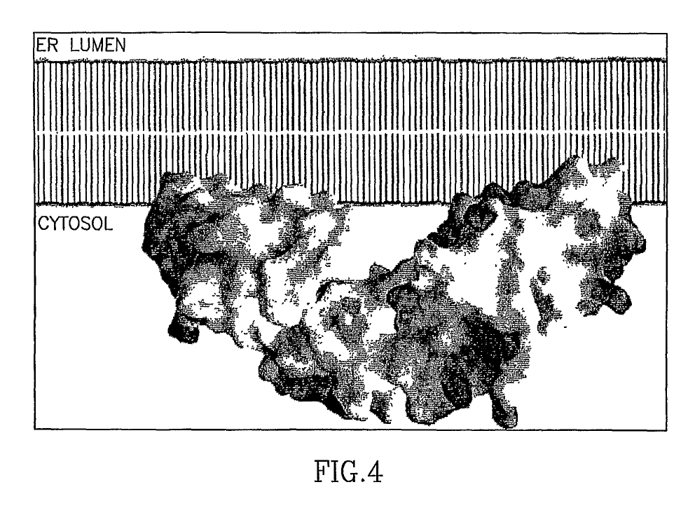

Helix H2 at the amino-terminus of the C-terminal domain of NS2 is highly hydrophobic and solvent accessible. The H2 helices of each monomer lie roughly parallel to each other at the ‘top’ of the molecule, as seen in FIG. 1A. Therefore, H2 of each NS2 molecule may be peripherally inserted into a cellular membrane. A model of the association of NS2 with the membrane of the endoplasmic reticulum (ER) is shown in FIG. 4. Peripheral membrane association of helix H2 would place the amino-termini of the two protein monomers in close proximity to the membrane. This is consistent with the putative topology model for the full-length NS2, in which the hydrophobic amino-terminal third of NS2 forms several transmembrane segments.

What is claimed is:

1. (canceled)

2. (canceled)

3. A computer readable data storage material encoded with computer readable data comprising structure coordinates of Table 1.

4. A crystallized C-terminal domain of an NS2 protein of hepatitis C virus, wherein said domain has secondary structural elements that include two alpha helices in the amino-terminal domain, designated as helices H1 and H2, and four beta strands in the carboxy-terminal domain, designated as b1, b2, b3, and b4.

5. The crystallized C-terminal domain of claim 4, wherein said crystallized C-terminal domain of an NS2 protein of hepatitis C virus has a papain-like cysteine protease activity and comprises catalytic residues histidine 143, glutamate 163, cysteine 184, leucine 217, or a combination thereof.

6. A method of identifying an HCV inhibitor in an inhibitor screening assay comprising:
(a) selecting a potential inhibitor by performing rational drug design with a three-dimensional structure determined for a crystallized C-terminal domain of an NS2 protein of a hepatitis C virus, wherein said selecting is performed in conjunction with computer modeling;
(b) contacting said potential inhibitor with a C-terminal domain of an NS2 protein of hepatitis C virus; and
(c) detecting an ability of said potential inhibitor for inhibiting infection or replication of a hepatitis C virus.

7. The method of claim 6, wherein said inhibitor interferes with the autoproteolytic activity of the C-terminal domain of an NS2 protein.

8. The method of claim 6, wherein said inhibitor interferes with dimerization of said C-terminal domains of NS2 proteins.

9. The method of claim 6, wherein said inhibitor interferes with membrane association of NS2 proteins of said virus.

10. A method of growing a crystallized C-terminal domain of an NS2 protein, comprising growing the crystal by vapor diffusion using a reservoir buffer containing 100 mM Tris pH 8.5, 0.8 M ammonium acetate, 0.25 M lithium chloride, and 12% (w/v) polyethylene glycol 3350, at 4° C.

11. A method for identifying a test compound that inhibits or prevents hepatitis C viral infection or pathogenesis, said method comprising:
a) contacting a cell in culture-infected with a hepatitis C virus in culture with a test compound, under conditions and for a time sufficient to permit the dimerization of NS2 proteins of said virus;
b) culturing a cell infected with hepatitis C virus in the absence of said agent, under conditions and for a time sufficient to permit the dimerization of said NS2 protein; and
c) comparing viral infection or pathogenic effects on cells cultured in (a) versus (b),
whereby a decrease or absence of viral infection or pathogenic effects on cells detected in (a) as compared to (b) indicates that the test compound inhibits or prevents hepatitis C viral infection or pathogenesis.

12. The method of claim 11, wherein said inhibitor interferes with association of NS2 with cellular membranes.

13. (canceled)

14. The method of claim 6, wherein said potential inhibitor is selected for interference with an active site comprising histidine 143, glutamate 163, cysteine 184, leucine 217, or combinations thereof.

15. (canceled)

16. (canceled)

17. (canceled)

18. (canceled)

19. (canceled)

20. (canceled)

21. (canceled)

22. The method of claim 14, wherein said potential agent is selected for interference with attractive forces between histidine 143 and glutamate 163 of a first monomer, and cysteine 184 and leucine 217 of a second monomer.

23. (canceled)

24. (canceled)

25. The crystallized C-terminal domain of an NS2 protein of hepatitis C virus of claim 4, wherein the crystal effectively diffracts X-rays for the determination of the atomic coordinates of the domain to a resolution of greater than 5.0 Angstroms, and wherein said crystal has a space group of P2₁, with unit cell dimensions of a=61.23 Å, b=67.27 Å, c=108.87 Å and α=γ=90°, β=105.82 Å.

26. The crystallized C-terminal domain of an NS2 protein of hepatitis C virus of claim 4, wherein the crystal effectively diffracts X-rays for the determination of the atomic coordinates of the domain to a resolution of greater than 5.0 Angstroms, and wherein said crystal has a space group of P2₁, with unit cell dimensions of a=109.81 Å, b=68.82 Å, c=125.16 Å, and α=γ=90°, β=105.88°.

27. The method of claim 6, wherein said ability for inhibiting infection or replication of a hepatitis C virus is assayed in vivo or in vitro.
